(19) United States
(12) Patent Application Publication
(10) Pub. No.: US 2023/0069964 A1
(43) Pub. Date: Mar. 9, 2023

(54) PRODUCTION OF LIQUEFIED PETROLEUM GAS (LPG) HYDROCARBONS FROM CARBON DIOXIDE-CONTAINING FEEDS

(71) Applicant: Gas Technology Institute, Des Plaines, IL (US)

(72) Inventors: Patrick LITTLEWOOD, Chicago, IL (US); Jim WANGEROW, Oak Park, IL (US); Terry MARKER, Park Ridge, IL (US); Andrew WOLEK, Evanston, IL (US)

(21) Appl. No.: 17/470,195

(22) Filed: Sep. 9, 2021

(51) Int. Cl. C10G 2/00; C01B 3/38; C01B 3/16; B01J 29/70; B01J 23/72

(52) U.S. Cl. CPC C10G 2/334; C10G 2/50; C01B 3/38; C01B 3/16; B01J 29/7007; B01J 23/72; C10G 2400/28; C10G 2300/70; C10G 2300/4081; C01B 2203/062; C01B 2203/0283; C01B 2203/0227

(57) ABSTRACT

Pathways are disclosed for the production of liquefied petroleum gas (LPG) products comprising propane and/or butane, and in certain cases renewable products having non-petroleum derived carbon. In particular, a gaseous feed mixture comprising CO₂ in combination with CH₄ and/or H₂ is converted by reforming and/or reverse water-gas shift (RWGS) reactions, further in combination with LPG synthesis. A preferred gaseous feed mixture comprises biogas or otherwise a mixture of CO₂ and H₂ that is not readily upgraded using conventional processes. Catalysts described herein have a high activity for reforming (including dry reforming) of CH₄, as well as simultaneously catalyzing RWGS. These attributes improve the management of CO₂ that is input to the disclosed processes, particularly in those utilizing recycle operation to increase overall CO₂ conversion. Economics of small scale operations may be improved, if necessary, using an electrically heated reforming reactor in the first or initial reforming stage or RWGS stage.

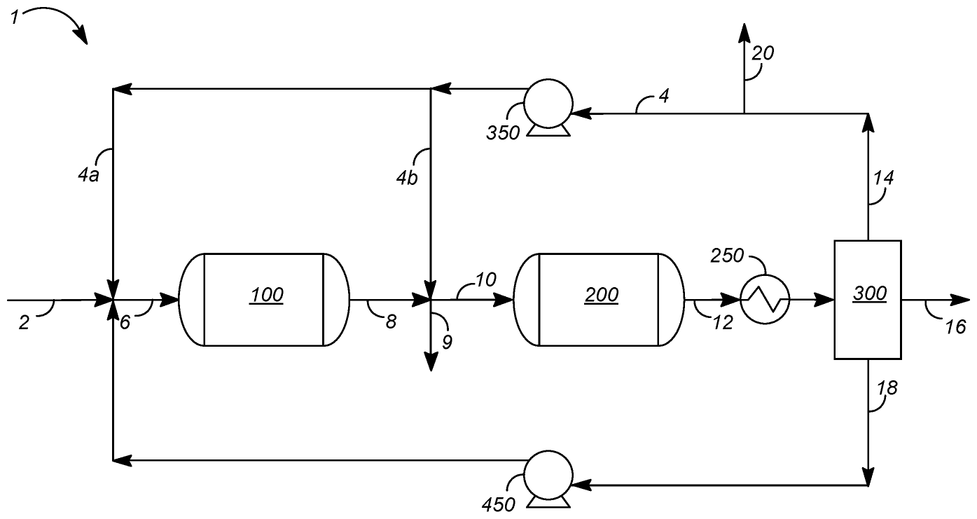

### FIELD OF THE INVENTION

Aspects of the invention relate to processes and associated catalysts for producing, from gaseous feed mixtures comprising carbon dioxide (CO₂), products comprising propane and/or butane, for example those having a composition approximating that of liquefied petroleum gas (LPG). Representative processes utilize at least (i) one or both reactions of reforming and reverse water-gas shift (RWGS), in combination with (ii) LPG synthesis. Other aspects relate more broadly to the conversion of synthesis gas, optionally comprising CO₂, to LPG.

### DESCRIPTION OF RELATED ART

The ongoing search for alternatives to crude oil, as a conventional source of carbon for hydrocarbon products, is increasingly driven by a number of factors. These include diminishing petroleum reserves, higher anticipated energy demands, and heightened concerns over greenhouse gas (GHG) emissions from sources of non-renewable carbon. Hydrocarbon products of greatest industrial significance and interest, in terms of having their carbon content replaced with non-petroleum derived carbon, include transportation and heating fuels as well as precursors for specialty chemicals. The particular hydrocarbons propane and/or butane are present in many of these products, a common example of which is liquefied petroleum gas (LPG).

Carbon dioxide (CO₂) is a major contributor to GHG emissions and is found in gases generated from combustion as performed in engines, electricity production, and both commercial and residential heating. In general, a great number of small- and large-scale processes produce waste gases containing CO₂ that is derived from the crude oil-based hydrocarbon products described above. In some cases, CO₂ may be obtained as a component of a mixture of gases including hydrogen (H₂) and/or methane (CH₄), in which the CO₂ may or may not be a combustion product. Examples of such mixtures include industrial off gases obtained from the production of H₂ by the reforming of CH₄, in which the CO₂ is used as a reactant (in the case of dry reforming) and/or is generated by the water-gas shift reaction. In addition, sources of natural gas, while predominantly methane, may also include a significant content of CO₂ that is extracted in this resource. Other gaseous mixtures of CO₂ with CH₄ include those in which the latter component is a renewable resource, such as in the specific case of (i) biogas obtained from anaerobic bacterial digestion of biowastes or from wastewater treatment, (ii) gaseous products of biomass conversion (e.g., biomass gasification, pyrolysis, or hydropyrolysis, such as in the case of supercritical water gasification of biomass), (iii) landfill gases, or (iv) gaseous products of the electrochemical reduction of carbon dioxide.

In view of its abundance in natural gas reserves and oil-associated gases, methane has become the focus of a number of possible synthesis routes. Currently, natural gas is the most underutilized of fossil resources, and it is frequently flared (combusted) in large quantities, particularly in the case of “stranded” natural gas or other sources that are too isolated and/or lacking in quantity, rendering their transport to large-scale processing facilities an uneconomical proposition. In addition, fracking technology has resulted in decreasing prices of natural gas in the U.S., with an increasing supply of this resource globally. Moreover, methane is one of the most common products that can be produced from renewable resources, and particularly those obtained from the processing of biowastes and biomass, as well as other resources as noted above. Therefore, the conversion of methane, and especially methane that is obtained from renewable carbon sources such as biowaste, represents an area of considerable interest for development on the industrial scale with favorable economics.

A key commercial process for converting methane into fuels involves a first conversion step to produce synthesis gas (syngas), followed by a second, downstream Fischer-Tropsch (FT) conversion step. With respect to the first conversion step, upstream of FT, known processes for the production of syngas from methane include partial oxidation reforming and autothermal reforming (ATR), based on the exothermic oxidation of methane with oxygen. Steam methane reforming (SMR), in contrast, uses steam as the oxidizing agent, such that the thermodynamics are significantly different, not only because the production of steam itself can require an energy investment, but also because reactions involving methane and water are endothermic. More recently, it has also been proposed to use carbon dioxide as the oxidizing agent for methane, such that the desired syngas is formed by the reaction of carbon in its most oxidized form with carbon in its most reduced form, according to:

CH₄+CO₂→2CO+2H₂.

This reaction has been termed the “dry reforming” of methane, and because it is highly endothermic, thermodynamics for the dry reforming of methane are less favorable compared to ATR or even SMR. However, the stoichiometric consumption of one mole of carbon dioxide per mole of methane has the potential to reduce the overall carbon footprint of liquid fuel production, providing a “greener” consumption of methane. This CO₂ consumption rate per mole of feed increases in the case of reforming higher hydrocarbons (e.g., C₂-C₆ paraffins), which may be desired, for example, if hydrogen production (e.g., for refinery processes) is the objective. In any event, the thermodynamic barrier remains a major challenge and relates to the fact that CO₂ is completely oxidized and very stable, such that significant energy is needed for its activation as an oxidant. In view of this, a number of catalyst systems have been investigated for overcoming the activation energy barrier for the dry reforming of methane, and these are summarized, for example, in a review by Lavoie (FRONTIERS IN CHEMISTRY (November 2014), Vol. 2 (81): 1-17), identifying heterogeneous catalyst systems as being the most popular in terms of catalytic approaches for carrying out this reaction.

Whereas nickel-based catalysts have shown effectiveness in terms of lowering the activation energy for the above dry reforming reaction, a high rate of carbon deposition (coking) of these catalysts has also been reported in Lavoie. The undesired conversion of methane to elemental carbon can proceed through methane cracking (CH₄→C+2H₂) or the Boudouard reaction (2CO→C+CO₂) at the reaction temperatures typically required for the dry reforming of methane. More recently, other types of catalysts, including those comprising noble metals on a ceria-containing support, have been described in U.S. Pat. Nos. 10,738,247; 10,906,808; US 2020/0087144; and US 2020/0087576, assigned to Gas Technology Institute (Des Plaines, Ill.). Such catalysts have been demonstrated to exhibit high activity and stability (low coking rate) in reforming based on CO₂ alone or a combination of CO₂ and steam. In addition, the high tolerance to sulfur-bearing contaminants (e.g., H₂S), exhibited by these catalysts, can further improve process economics in terms of lowering costs normally associated with feed pretreatment.

With respect to the second step involving FT conversion, synthesis gas containing a mixture of hydrogen and carbon monoxide (CO) is subjected to successive cleavage of C—O bonds and formation of C—C bonds with the incorporation of hydrogen. This mechanism provides for the formation of hydrocarbons, and particularly straight-chain alkanes with a distribution of molecular weights that can be controlled to some extent by varying the FT reaction conditions (temperature and feed CO:H₂ ratio) and catalyst properties. Such properties include pore size and other characteristics of the support material. The choice of catalyst can impact FT product yields in other respects. For example, iron-based FT catalysts tend to produce more oxygenates, whereas ruthenium as the active metal tends to produce exclusively paraffins. The reaction pathways of FT synthesis follow a statistical kinetic model, which leads to hydrocarbons having an Anderson-Schultz-Flory distribution of their carbon numbers. In the case of targeting the C₃ and C₄ hydrocarbons, i.e., propane and butane, this generally involves operating in a low conversion regime with a significant co-production of methane and ethane. Higher conversions, on the other hand, generate C₅⁺ hydrocarbons that are liquid at room temperature. Other potential routes for the production of LPG hydrocarbons from syngas are described by K. Asami et al. (STUDIES IN SURFACE SCIENCE AND CATALYSIS 147 (2004) 427-432); Q. Zhang et al. (FUEL PROCESSING TECHNOLOGY 85 (2004) 1139-1150); and Q. Ge et al. (JOURNAL OF MOLECULAR CATALYSIS A: CHEMICAL 278 (2007) 215-219.

In terms of known pathways offering potential conversion routes to LPG hydrocarbons from methane, and desirably renewable methane such as that present in biogas, improvements are needed in a number of areas. These include reaction product selectivity and yield and/or the management of CO₂ that is often present in gaseous feed mixtures or that can otherwise result from the prevailing process chemistry (e.g., via water-gas shift). Overall, the state of the art would benefit from technologies for the efficient conversion of industrially available gaseous mixtures containing CO₂ and other important reactants such as H₂ and/or CH₄, to products comprising propane and/or butane, for example those having a composition approximating that of liquefied petroleum gas (LPG). With respect to the practical impact of such technologies, a current objective of a number of countries around the world is to reduce deforestation and the generation of pollution, both of which are associated with the burning of wood for heating and cooking. However, because of the remoteness of many locations and the associated, long transportation routes, petroleum-derived LPG is priced at a premium and therefore not considered a viable alternative to wood. Accordingly, a number of significant advantages could be gained by efficiently obtaining LPG hydrocarbons from renewable sources and other readily available gaseous mixtures. These advantages include freedom from the need to import petroleum-derived LPG, a reduction in GHG emissions, improvement in air quality, and the potential stimulation of local economies, particularly in poorer regions.

### SUMMARY OF THE INVENTION

Aspects of the invention are associated with the discovery of novel pathways for the production of liquefied petroleum gas (LPG) products comprising propane and/or butane, and in certain cases renewable LPG products, i.e., in which some or all (e.g., at least about 70%) of their carbon content (whether expressed on a wt-% or mole-% basis) is renewable carbon that is not derived from petroleum. Advantageously, whether or not the carbon content is renewable carbon, at least a portion (e.g., at least about 20%, at least about 30%, or at least about 40%), of the total carbon content of representative LPG products described herein may be derived from CO₂, for example being present initially in a gaseous feed mixture or a fresh makeup feed. In the case of a renewable carbon content that is also derived from CO₂, such CO₂ may be obtained, for example, from biogas as a product of bacterial digestion (i.e., such CO₂ is originally contained in biogas) or from gaseous products of biomass conversion, such as a biomass gasifier product (i.e., such CO₂ is originally contained in a gasifier product). In the case of a non-renewable carbon content that is derived from CO₂, such CO₂ (e.g., present initially in a gaseous feed mixture or a fresh makeup feed) may be obtained, for example, as a fossil fuel combustion product or a fossil fuel reforming product. In either case, it can be appreciated that CO₂ used to provide at least a portion of the total carbon content is beneficially utilized as LPG, rather than being directly released into the atmosphere.

Further aspects of the invention are associated with the discovery that common sources of CO₂, and especially gaseous mixtures of CO₂ with either or both of CH₄ and H₂, can be used efficiently as feeds in producing LPG products. Importantly, the whole feed and therefore all of these components may be reactants in one or both reactions of reforming and reverse water-gas shift (RWGS) to produce a synthesis gas intermediate. Such reaction(s) are used in combination with further conversion by LPG synthesis, to obtain propane and/or butane in an LPG product. In the case of a gaseous feed mixture or a fresh makeup feed comprising both CH₄ and CO₂, e.g., a gaseous mixture or a fresh makeup feed that is biogas or that comprises biogas, these components may be reacted in a reforming stage, according to the dry reforming reaction above, to produce a synthesis gas intermediate comprising H₂ and CO (i.e., an H₂/CO mixture). This intermediate may, in turn, be converted to the LPG product via LPG synthesis. In the case of a gaseous feed mixture or a fresh makeup feed comprising both H₂ and CO₂, e.g., a gaseous mixture or fresh makeup feed that is or comprises an industrial off gas, such as a “PSA tail gas” (or “PSA off gas”), these components may be reacted according to the RWGS reaction to produce a synthesis gas intermediate for conversion to an LPG product as described above. As is known in the art, a PSA tail gas is a byproduct obtained from the production of H₂ by the reforming of CH₄. Simultaneously with the RWGS reaction, CH₄ and CO₂ components of the gaseous feed mixture or fresh makeup feed (e.g., as components of a PSA tail gas or other industrial off gas) may be reacted according to the dry reforming reaction above, thereby adding to the yield of H₂ and CO in the synthesis gas intermediate.

Accordingly, other aspects of the invention are associated with the discovery that catalysts described herein, having a high activity for catalyzing the reforming (including dry reforming) of CH₄ are likewise effective, under the same conditions, for catalyzing the RWGS reaction. These attributes of such catalysts are therefore advantageous in producing LPG products, particularly from a gaseous feed mixture or a fresh makeup feed, as described herein, comprising CO₂ together with CH₄ and/or H₂, all of which components may be beneficially utilized as reactants in these reactions. Importantly, RWGS activity, optionally in combination with recycle of an H₂/CO₂-enriched fraction of (or H₂/CO₂-enriched fraction separated from) the LPG synthesis effluent as described herein, allows for the effective management/conversion of CO₂ that is present in a gaseous feed mixture or a fresh makeup feed, such as in a significant amount (e.g., at least about 20 mol-%). Such mixture or feed may otherwise be difficult to monetize and/or may conventionally be combusted for heating value. In the case reforming and/or RWGS reactions (in a reforming stage or an RWGS stage), followed by LPG synthesis, further integration with the recycle of an H₂/CO₂-enriched fraction can substantially improve overall LPG yield (e.g., based on carbon in the fresh makeup feed) and overall process economics.

Particular aspects of the invention are associated with advantages that may be attained from the recycle of H₂ and CO₂, present in an LPG synthesis effluent, back either to the first stage (e.g., a reforming stage, such as a reforming/RWGS stage, or an RWGS stage) or to the second, LPG synthesis stage of the process. For example, it has been determined that the recycle of H₂ and CO₂ in combination, particularly to the LPG synthesis stage (e.g., by combining of H₂ and CO₂, separated from the LPG synthesis effluent, with the synthesis gas intermediate or a portion thereof that is obtained as a product of the first stage) results in a surprising increase in selectivity of the LPG synthesis reaction to LPG hydrocarbons, namely C₃ and C₄ hydrocarbons. To the extent that the per-pass CO conversion in the LPG synthesis stage may be optimized (e.g., increased) through adjustments to LPG synthesis conditions, such as by decreasing space velocity to increase reactant residence time and/or increasing pressure to increase reactant concentrations, the observed, increase in selectivity corresponds to an increase in the per-pass product yield, relative to that obtained in a baseline process with the same CO conversion level but without recycle. In this regard, as is recognized by those skilled in the art, even modest increases in selectivity and/or per-pass yield will generally translate to very significant economic benefits on a commercial scale. Such benefits may be attributed, for example, to a reduced formation of undesired byproducts and/or reduced recycle gas requirements.

Additionally, in some embodiments, for example those involving the processing of gaseous feed mixtures on a relatively small scale, the use of an electrically heated reforming reactor in the first or initial stage (e.g., a reforming stage or an RWGS stage) to perform one or both of these reactions may further improve processing efficiency and equipment compactness, leading to reduced costs. Small scale operations may involve, for example, the processing of gaseous feed mixtures or fresh makeup feeds obtained from lower capacity biogas production facilities or stranded gas reserves. An electrically heated reforming reactor may include one or more resistive or inductive heating elements for the control of heat input into a bed of reforming/RWGS catalyst as described herein. Representative electrically heated reforming reactors thereby provide localized and responsive bed temperature control, and examples of these are described in co-pending U.S. provisional application Ser. No. 63/107,537, hereby incorporated by reference in its entirety.

Particular embodiments of the invention are directed to processes for producing LPG products comprising propane and/or butane, as well as LPG products obtained from such processes. These include LPG products in which at least a portion (e.g., at least about 70% on a weight or molar basis) of the carbon content of the propane and/or butane contained in these products is renewable carbon. Representative processes comprise a first stage for carrying out reforming and/or RWGS reactions, i.e., in a reforming stage, in an RWGS stage, or in a reforming/RWGS stage, on a gaseous feed mixture or on a fresh makeup feed. This is followed by a second stage of converting at least a portion of a synthesis gas intermediate produced in the first stage and comprising both H₂ and CO (i.e., an H₂/CO mixture). In particular, this intermediate, or portion thereof, is converted in an LPG synthesis stage to propane and/or butane that is contained in the LPG product. According to specific embodiments, in the first stage, a gaseous feed mixture or a fresh makeup feed comprising predominantly (i) CH₄ and CO₂ or (ii) H₂ and CO₂ is contacted with a catalyst as described herein (e.g., a reforming/RWGS catalyst) to produce the synthesis gas intermediate. In the second stage, the conversion of the synthesis gas intermediate or portion thereof to LPG may proceed through a methanol synthesis reaction mechanism whereby, for example, methanol produced from H₂ and CO in the synthesis gas intermediate is dehydrated to LPG hydrocarbons and water. In view of the hydrogen requirement for methanol synthesis and dehydration, the synthesis gas intermediate, or portion thereof that is used for LPG synthesis in the second stage, may have an H₂:CO molar ratio of at least about 2.0, such as from about 2.0 to about 2.5. Such molar ratios may be obtained from the first stage, optionally following an adjustment of the H₂:CO molar ratio.

Conversion of the synthesis gas intermediate to the LPG product may comprise contacting this intermediate or portion thereof with an LPG synthesis catalyst system having activities for both methanol synthesis and dehydration. This catalyst system may comprise, for example, a catalyst mixture comprising both a methanol synthesis catalyst and a dehydration catalyst, such as in the case of separate compositions (e.g., each in the form of a separate particles) of these catalysts. The catalyst system may alternatively, or in combination, comprise a bi-functional catalyst having both a methanol synthesis-functional constituent and a dehydration-functional constituent. In the case of either a catalyst mixture or a bi-functional catalyst, (i) the respective methanol synthesis catalyst or methanol synthesis-functional constituent may comprise one or more methanol synthesis-active metals selected from the group consisting of Cu, Zn, Al, Pt, Pd, and Cr, and/or (ii) the respective dehydration catalyst or dehydration functional constituent may comprise a zeolite or non-zeolitic molecular sieve.

Further embodiments of the invention are directed to processes for producing an LPG product from a synthesis gas comprising H₂ and CO, for example a synthesis gas intermediate or an LPG synthesis feed obtained following one or more intervening operations performed on this intermediate, as described herein. More broadly, any source of synthesis gas may be used as an LPG synthesis feed in representative LPG synthesis processes, including LPG synthesis feeds having an H₂:CO molar ratio that is representative of a synthesis gas intermediate, as described herein. The synthesis gas intermediate or LPG synthesis feed may be produced by reforming and/or RWGS reactions, as described herein. However, more broadly, LPG synthesis processes according to some embodiments do not require a specific source of synthesis gas, and such embodiments are directed to such processes (e.g., processes comprising a stage of LPG synthesis, such as in the case of single stage processes) that do not necessarily require a given upstream conversion step (e.g., a reforming stage as described herein). Representative processes comprise contacting, as an LPG synthesis feed, broadly any source of synthesis gas comprising H₂ and CO (e.g., in a combined amount of greater than about 50 mol-%), or more specifically any particular synthesis gas intermediate or LPG synthesis feed as described herein, with an LPG synthesis catalyst system as described herein, to convert H₂ and CO, and optionally CO₂, in the synthesis gas to hydrocarbons, including propane and/or butane that are provided in an LPG product. In some cases, the LPG synthesis feed may further comprise CO₂, for example in an amount of at least about 5 mol-% (e.g., from about 5 mol-% to about 50 mol-%), at least about 10 mol-% (e.g., from about 10 mol-% to about 35 mol-%), or at least about 15 mol-% (e.g., from about 15 mol-% to about 30 mol-%). In such cases, the balance of the LPG synthesis feed may be, or may substantially be, H₂ and CO in combination, for example in an H₂:CO molar ratio that is representative of a synthesis gas intermediate, as described herein. Particularly advantageous results may be obtained in the case of LPG synthesis feeds comprising CO₂, as would be apparent to those skilled in the art having knowledge of the present disclosure.

Other particular embodiments are directed to processes described above, according to which biogas is converted to the LPG product, i.e., the gaseous feed mixture or the fresh makeup feed is, or comprises, biogas. Advantageously, biogas provides a readily available gaseous feed mixture or fresh makeup feed, or portion of either of these, which comprises predominantly CH₄ and CO₂. Importantly, an abundance of biogas may be present in locations remote from sources of conventional LPG, such that particular processes involving the processing of biogas may represent an economically efficient alternative for obtaining propane and/or butane that may be used, for example, in heating (e.g., cooking) applications. Moreover, the carbon content of propane and/or butane of LPG products made in this manner is derived from CH₄ and CO₂ originating from organic waste, i.e., the carbon content is renewable. Representative processes according to these particular embodiments comprise, in a reforming stage (and possibly, but not necessarily, a reforming/RWGS stage), contacting biogas (or a gaseous feed mixture or fresh makeup feed comprising biogas) with a reforming/RWGS catalyst to produce a synthesis gas intermediate comprising an H₂/CO mixture. The processes may further comprise converting at least a portion of the synthesis gas intermediate to the LPG product, for example through a methanol synthesis reaction mechanism as described herein.

These and other embodiments, aspects, and advantages relating to the present invention are apparent from the following Detailed Description.

### BRIEF DESCRIPTION OF THE DRAWING

A more complete understanding of the exemplary embodiments of the present invention and the advantages thereof may be acquired by referring to the following description in consideration of the accompanying FIGURE, which provides a flow scheme of a process for producing an LPG product.

The FIGURE should be understood to present an illustration of a process and certain principles involved. In order to facilitate explanation and understanding, this FIGURE provides a simplified overview, with the understanding that the depicted elements are not necessarily drawn to scale. Valves, instrumentation, and other equipment and systems not essential to the understanding of the various aspects of the invention are not shown. As is readily apparent to one of skill in the art having knowledge of the present disclosure, processes for the production of LPG hydrocarbons via the reactions of reforming and/or RWGS, may have alternative configurations and elements that are governed by specific operating objectives, but which alternatives are nonetheless within the scope of the invention.

### DETAILED DESCRIPTION

The expressions “wt-%” and “mol-%,” are used herein to designate weight percentages and molar percentages, respectively. The expressions “wt-ppm” and “mol-ppm” designate weight and molar parts per million, respectively. For ideal gases, “mol-%” and “mol-ppm” are equal to percentages by volume and parts per million by volume, respectively. In some cases, a percentage, “%,” is given with respect to values that are the same, whether expressed as a weight percentage or a molar percentage. For example, (i) the percentage of the feed carbon content that forms propane and/or butane of the LPG product, or (ii) the percentage of the carbon content of the LPG product that is renewable carbon or carbon derived from CO₂, has the same value, whether expressed as a weight percentage or a molar percentage.

The term “substantially,” as used herein, refers to an extent of at least 95%. For example, the phrase “substantially all” may be replaced by “at least 95%.”

A “gaseous feed mixture” as described herein may be representative of the entirety of the feed that is fed or input, e.g., that is input in one stream, or in two or more separate or combined streams, to a reactor used in a first stage of the process, namely the reforming stage or the RWGS stage (e.g., a reforming/RWGS stage). In a particular embodiment according to which recycle is utilized, the gaseous feed mixture may be provided to such reactor or reaction stage as a combination of (i) a fresh makeup feed and (ii) an H₂/CO₂-enriched fraction of an LPG synthesis effluent, as described herein. That is, the gaseous feed mixture may comprise (i) and (ii), such that a fresh makeup feed may, according to particular embodiments associated with any “gaseous feed mixture” described herein, be a portion of such gaseous feed mixture. Any description of an “H₂/CO₂-enriched fraction” can, according to alternative embodiments, refer more specifically to “a portion” of such “H₂/CO₂-enriched fraction,” for example a recycle portion of this fraction, or even a part of such recycle portion, consistent with the further disclosure below. For example, a purge stream, sampling streams, etc. may be removed from the H₂/CO₂-enriched fraction of an LPG synthesis effluent, leaving only a recycle portion of such fraction to be returned to the process, such as to the first stage (e.g., a reforming stage, such as a reforming/RWGS stage, or an RWGS stage) and/or the LPG synthesis stage, optionally with different parts of this recycle portion being routed to different stages. In view of the above description, and further description herein relating to recycle operation, the gaseous feed mixture may comprise a fresh makeup feed and/or a recycle portion of an H₂/CO₂-enriched fraction (or even a part of such fraction) that is separated from an LPG synthesis effluent.

Likewise, an “LPG synthesis feed” as described herein may be representative of the entirety of the feed that is fed or input, e.g., that is input in one stream, or in two or more separate or combined streams, to a reactor used in a second stage of the process, namely the LPG synthesis stage. In a particular embodiment according to which recycle is utilized, the LPG synthesis feed may be provided to such reactor or reaction stage as a combination of (i) a synthesis gas intermediate or portion thereof (e.g., withdrawn directly from a reactor used in a first stage of the process) and (ii) an H₂/CO₂-enriched fraction of an LPG synthesis effluent, as described herein. That is, the LPG synthesis feed may comprise (i) and (ii), such that a synthesis gas intermediate or portion thereof may, according to particular embodiments, be a portion of such LPG synthesis feed. As noted above, an “H₂/CO₂-enriched fraction” can, according to alternative embodiments, refer more specifically to “a portion” of such “H₂/CO₂-enriched fraction,” for example a recycle portion of this fraction, or even a part of such recycle portion, consistent with the further disclosure below.

In representative processes described herein, a first (upstream) or initial stage may be referred to as “a reforming/RWGS stage” to indicate that both reforming and reverse water-gas shift (RWGS) reactions occur to some extent. Reforming, as understood in the art and in the context of the present disclosure, refers to the reaction of CH₄ with an oxidant to produce H₂ and CO (synthesis gas), with the oxidant being preferably CO₂, but possibly comprising any one or more of CO₂, H₂O, and O₂. The RWGS reaction is understood in the art as the following:

H₂+CO₂→H₂O+CO.

In broader embodiments, the first or initial stage may be “a reforming stage,” in which the reforming of CH₄ occurs as noted above, whereas the RWGS reaction does not necessarily occur. In other broader embodiments, the first or initial stage may be “an RWGS stage,” in which the RWGS reaction occurs as noted above, whereas the reforming of CH₄ does not necessarily occur. For example, in the case of a gaseous feed mixture comprising CH₄ and CO₂, the first stage may be a reforming stage in which these components react to produce synthesis gas. Typically, however, at least some H₂ of the synthesis gas, and present in the reaction mixture, reacts with CO₂, also present in the reaction mixture, according to the RWGS reaction, such that the reforming stage may be more specifically characterized as “a reforming/RWGS stage.” In the case of a gaseous feed mixture comprising H₂ and CO₂, the first stage may be an RWGS stage in which these components react as indicated above. It can be appreciated, therefore, that the first or initial stage may be either a reforming stage or an RWGS stage, in the case of a gaseous feed mixture comprising, together with CO₂, either CH₄ or H₂, respectively. In the case of any gaseous feed mixture comprising CH₄ together with CO₂ (e.g., comprising CH₄, CO₂, and H₂) the first or initial stage may be a reforming/RWGS stage.

In general, the first, reforming stage or RWGS stage is followed by a second (downstream) stage of LPG synthesis, which utilizes at least a portion of the synthesis gas intermediate produced in the first stage, optionally following one or more intervening operations as described herein. According to representative embodiments, the first and second stages may be the only stages of the process involving reactions and/or the use of catalysts or catalyst systems for carrying out these reactions. Optionally, processes may include other reaction stages, i.e., the designation of the reforming stage or the RWGS stage as the “first” stage and the designation of the LPG synthesis stage as the “second” stage does not preclude the possibility of one or more other reaction stages, prior to the first stage, between the first and second stages, and/or following the second stage. For example, an additional reaction stage may be used to perform the water-gas shift reaction for the generation of H₂ and CO₂.

### Gaseous Feed Mixtures

Exemplary processes, for producing an LPG product comprising propane and/or butane, include (a) in a reforming stage or an RWGS stage, contacting a gaseous feed mixture with a reforming/RWGS catalyst to produce a synthesis gas intermediate comprising an H₂/CO mixture; and (b) converting the synthesis gas intermediate to the LPG product, such as via methanol synthesis and dehydration. Representative gaseous feed mixtures comprise predominantly (i) CH₄ and CO₂ or (ii) H₂ and CO₂, with the term “predominantly” referring to these gaseous feed mixtures comprising (i) CH₄ and CO₂ in a combined amount of at least 50 mol-%, or (ii) H₂ and CO₂ in a combined amount of at least 50 mol-%. In more specific embodiments, gaseous feed mixtures comprise (i) CH₄ and CO₂ in a combined amount of at least 75 mol-%, at least about 90 mol-%, or at least about 95 mol-%, or (ii) H₂ and CO₂ in a combined amount of at least 75 mol-%, at least about 90 mol-%, or at least about 95 mol-%. According to other embodiments, representative gaseous feed mixtures may comprise CH₄, CO₂, and H₂ in a combined amount of at least 50 mol-%, at least about 75 mol-%, at least about 90 mol-%, or at least about 95 mol-%. Alternatively, or in combination with any of the features described herein, representative gaseous feed mixtures may comprise little or no amounts of other components. For example, in the case of a gaseous feed mixture comprising predominantly (i) CH₄ and CO₂, such gaseous feed mixture may comprise H₂ in an amount of less than about 25 mol-%, less than about 10 mol-%, less than about 5 mol-%, or less than about 1 mol-%. In the case of a gaseous feed mixture comprising predominantly (ii) H₂ and CO₂, such gaseous feed mixture may comprise CH₄ in an amount of less than about 25 mol-%, less than about 10 mol-%, less than about 5 mol-%, or less than about 1 mol-%. Any gaseous feed mixture described herein may comprise oxygen-containing components other than CO₂, for example, one or more of CO, H₂O, and O₂ in a respective amount (individually), or in a combined amount, of less than about 10 mol-%, less than about 5 mol-%, or less than about 1 mol-%. In such cases, due to the limited presence, or absence, of oxidants other than CO₂, any reforming of CH₄ that occurs in a reforming stage or in a reforming/RWGS stage may be substantially, or entirely, dry reforming and/or may be substantially, or entirely, unaccompanied by partial oxidation.

In the case of the gaseous feed mixture comprising predominantly (i) CH₄ and CO₂, step (a) may be a reforming stage, and optionally a reforming/RWGS stage, as described above, according to which, in either case, H₂ and CO in the H₂/CO mixture of the synthesis gas intermediate may be produced from the reaction of CH₄ and CO₂. In the case of the gaseous feed mixture comprising predominantly (ii) H₂ and CO₂, step (a) may be an RWGS stage, and optionally a reforming/RWGS stage, as described above. If step (a) is an RWGS stage, H₂ in the H₂/CO mixture of the synthesis gas intermediate may be H₂ that is unreacted, or that represents an equilibrium amount, in the RWGS reaction of H₂ and CO₂ as described above, whereas CO in this H₂/CO mixture may be CO that is produced in the RWGS reaction. If step (a) is a reforming/RWGS stage, the gaseous feed mixture comprising predominantly (ii) H₂ and CO₂ may further comprise CH₄. Therefore, H₂ and CO in the H₂/CO mixture of the synthesis gas intermediate may be produced from the reaction of CH₄ and CO₂. It may be further appreciated that, whether or not the gaseous feed mixture comprises CH₄ that allows H₂ to be produced from reforming, the H₂ and CO in the H₂/CO mixture of the synthesis gas intermediate may represent equilibrium amounts in the RWGS reaction. In specific embodiments in which the gaseous feed mixture comprises CH₄, the H₂ and CO in the H₂/CO mixture of the synthesis gas intermediate may represent equilibrium amounts in combined reforming and RWGS reactions. To the extent that, in a reforming stage or reforming/RWGS stage, CH₄ and CO₂ are reacted according to the dry reforming reaction described above, the reaction of CH₄ with one or both of the other oxidants H₂O and O₂ may also produce H₂ and/or CO in the H₂/CO mixture of the synthesis gas intermediate. For example, these other oxidants may also be present in the gaseous feed mixture, or, alternatively, H₂O may be present in the reaction mixture (although not necessarily present in the gaseous feed mixture) as a product of the RWGS reaction.

The gaseous feed mixture, or at least components of this mixture (e.g., CO₂, CH₄, and/or H₂), may be obtained from a wide variety of sources. Advantageously, such sources include waste gases that are regarded as having little or no economic value, and that may otherwise contribute to atmospheric CO₂ levels. For example, the gaseous feed mixture may be, or may comprise, an industrial process waste gas that is obtained from a steel manufacturing process or a non-ferrous product manufacturing process. Other processes from which all or a portion of the gaseous feed mixture may be obtained include petroleum refining processes (e.g., processes producing refinery off gases), renewable hydrocarbon fuel (biofuel) production processes (e.g., pyrolysis processes, such as hydropyrolysis processes, or a fatty acid/triglyceride hydroconversion processes), biomass and coal (e.g., lignocellulose and char) gasification processes, electric power production processes, carbon black production processes, ammonia production processes, other chemical (e.g., methanol) production processes, and coke manufacturing processes. In some cases, the gaseous feed mixture may be, or may comprise, (i) a wellhead gas comprising methane or (ii) a gaseous product of the electrochemical reduction of carbon dioxide.

A particular gaseous feed mixture of interest is biogas, which is understood to include (i) products of anaerobic bacterial digestion of biowastes, as well as (ii) landfill gases. Typically, biogas contains methane in an amount from about 35 mol-% to about 90 mol-% (e.g., about 40 mol-% to about 80 mol-% or about 50 mol-% to about 75 mol-%) and CO₂ in an amount from about 10 mol-% to about 60 mol-% (e.g., about 15 mol-% to about 55 mol-% or about 25 mol-% to about 50 mol-%). The gases N₂, H₂, H₂S, and O₂ may be present in minor amounts (e.g., in a combined amount of less than 20 mol-%, or less than 10 mol-%). In some embodiments, therefore, a gaseous feed mixture may be, or may comprise, biogas or other gas having these composition features.

Another gaseous feed mixture of interest is natural gas comprising methane in an amount from about 65 mol-% to about 98 mol-% (e.g., about 70 mol-% to about 95 mol-% or about 75 mol-% to about 90 mol-%) and CO₂ in an amount from about 3 mol-% to about 35 mol-% (e.g., about 5 mol-% to about 30 mol-% or about 10 mol-% to about 25 mol-%). Other hydrocarbons (e.g., ethane and propane), as well as nitrogen, may be present in minor amounts. Of particular interest is stranded natural gas, which, using known processes, is not easily converted to a synthesis gas intermediate in an economical manner. In some embodiments, therefore, a gaseous feed mixture may be, or may comprise, natural gas, for example comprising a relatively high amount of CO₂, such as at least about 10 mol-% or even at least about 25 mol-%.

A further gaseous feed mixture of interest is a hydrogen-depleted PSA tail gas, for example obtained from a hydrogen production processes involving steam methane reforming (SMR), as described above. This mixture may comprise (i) methane in an amount from about 5 mol-% to about 45 mol-% (e.g., about 10 mol-% to about 35 mol-% or about 15 mol-% to about 25 mol-%), (ii) CO₂ in an amount from about 20 mol-% to about 75 mol-% (e.g., about 25 mol-% to about 70 mol-% or about 35 mol-% to about 60 mol-%), and (iii) an H₂ in an amount from about 10 mol-% to about 45 mol-% (e.g., about 15 mol-% to about 40 mol-% or about 20 mol-% to about 35 mol-%). The balance of this stream may comprise predominantly water vapor and/or CO. In some embodiments, therefore, a gaseous feed mixture may be, or may comprise, a hydrogen-depleted PSA tail gas.

A further gaseous feed mixture of interest is a gaseous effluent from a biological (bacterial) fermentation that is integrated with a hydrogen production process. Such integrated fermentation processes are described, for example, in U.S. Pat. Nos. 9,605,286; 9,145,300; US 2013/0210096; and US 2014/0028598. Such gaseous effluent may comprise (i) methane in an amount from about 5 mol-% to about 55 mol-% (e.g., about 5 mol-% to about 45 mol-% or about 10 mol-% to about 40 mol-%), (ii) CO₂ in an amount about 5 mol-% to about 75 mol-% (e.g., about 5 mol-% to about 60 mol-% or about 10 mol-% to about 50 mol-%), and (iii) H₂ in an amount from about 5 mol-% to about 40 mol-% (e.g., about 5 mol-% to about 30 mol-% or about 10 mol-% to about 25 mol-%). The balance of this stream may comprise predominantly water vapor and/or CO. In some embodiments, therefore, a gaseous feed mixture may be, or may comprise, such gaseous effluent from fermentation.

In some embodiments, the compositions of gaseous feed mixtures as described herein may be representative of a combined composition of two or more streams being separately fed, or input, to a reactor used in the reforming stage or the RWGS stage. Separate streams may include, for example, fresh feed and/or recycle streams (e.g., a fresh makeup feed and/or an H₂/CO₂-enriched fraction as described herein, or a recycle portion of such fraction) or streams of one component, or enriched in one component (e.g., a CH₄-enriched stream), relative to the gaseous feed mixture. Any of the composition features described above with respect to a gaseous feed mixture can, according to alternative embodiments, apply to a fresh makeup feed that may be, for example, a portion of the gaseous feed mixture that is fed, or input, to a reactor used in the reforming stage or the RWGS stage, such as in the case of recycle operation.

### Reforming/RWGS Catalysts

As described above, an important aspect associated with the invention is the discovery that catalysts described herein can catalyze both the reforming (including dry reforming) of CH₄ and the RWGS reaction, to various extents that depend on the composition of the particular gaseous feed mixture or fresh makeup feed, as described herein, and particular reforming/RWGS conditions used. This provides considerable flexibility with respect to compositions of gaseous feed mixtures that may be processed into a synthesis gas intermediate using reforming and/or RWGS reactions. As used herein, the term “reforming/RWGS catalyst” refers to a catalyst having at least some activity for catalyzing reforming and/or at least some activity for catalyzing RWGS in an initial or upstream stage of the process, whether such stage may be characterized as a reforming stage or an RWGS stage. In preferred embodiments, such catalyst will catalyze both reactions to at least some extent, in a reforming/RWGS stage, given the gaseous feed mixture and conditions used.

Representative embodiments comprise contacting, in a reforming stage or an RWGS stage, a gaseous feed mixture as described herein with a reforming/RWGS catalyst. This contacting may be performed batchwise, but preferably is performed continuously, with a continuous flow of the gaseous feed mixture to one or more reactors (and preferably to a single reactor) used in this stage that contain the reforming/RWGS catalyst (e.g., such that this catalyst is disposed in a catalyst bed volume within the reactor). The reforming stage or the RWGS stage may therefore likewise include the continuous withdrawal from the reactor(s) of the synthesis gas intermediate comprising an H₂/CO mixture, i.e., the intermediate product comprising both H₂ and CO produced from reforming and/or RWGS reactions as described above.

Catalysts described herein exhibit a number of important advantages compared to conventional reforming catalysts, particularly in terms of tolerance to certain components that may be present in the gaseous feed mixture, such as C₂⁺ hydrocarbons (both paraffinic and olefinic) and/or H₂S or other sulfur-bearing components (e.g., mercaptans). Such characteristics reduce the significant pretreating requirements of conventional processes and thereby improve flexibility, in terms of economically producing the synthesis gas intermediate, even on a relatively small operating scale, from common process streams containing significant concentrations of such components. In some embodiments, any of the gaseous feed mixtures described herein may comprise, in addition to CO₂, CH₄, and/or H₂, one or both of (i) one or more C₂⁺ paraffinic hydrocarbons, such as ethane, propane, butane, pentane, and/or C₆⁺ paraffinic hydrocarbons and (ii) one or more C₂⁺ olefinic hydrocarbons, such as ethylene, propylene, butene, pentene, and/or C₆⁺ olefinic hydrocarbons. In one embodiment, the gaseous feed mixture may comprise one or more C₂⁺ paraffinic hydrocarbons, selected from the group consisting of ethane, propane, butane, pentane, and combinations of these. Any of these paraffinic hydrocarbons, or combination of paraffinic hydrocarbons, may be present, for example, in an amount, or total (combined) amount, of at least about 1 mol-% (e.g., from about 1 mol-% to about 35 mol-%), such as at least about 3 mol-% (e.g., from about 3 mol-% to about 20 mol-%). In another embodiment, the gaseous feed mixture may comprise one or more C₂⁺ olefinic hydrocarbons, selected from the group consisting of ethylene, propylene, butene, pentene, and combinations of these. Any of these olefinic hydrocarbons, or combination of olefinic hydrocarbons, may be present, for example, in an amount, or total (combined) amount, of at least about 0.3 mol-% (e.g., from about 0.3 mol-% to about 15 mol-%), such as at least about 1 mol-% (e.g., from about 1 mol-% to about 10 mol-%). In general, any one or more hydrocarbons other than CH₄ may be present in the gaseous feed mixture in an amount, or in a total (combined) amount, of at least about 3 mol-% (e.g., from about 3 mol-% to about 45 mol-%), such as at least about 5 mol-% (e.g., from about 5 mol-% to about 30 mol-%). In terms of their sulfur tolerance, reforming/RWGS catalysts described herein provide further advantages associated with the ability to process sulfur-containing gaseous feed mixtures, such as those comprising or being derived from natural gas that, depending on its source, may contain sulfur in the form of H₂S or other sulfur-bearing components. In general, the gaseous feed mixture may comprise at least about 1 mole-ppm (e.g., from about 1 mol-ppm to about 1 mol-%) total sulfur (e.g., present as H₂S and/or other sulfur-bearing components), such as at least about 3 mol-ppm (e.g., from about 3 mol-ppm to about 5000 mol-ppm) of total sulfur, at least about 10 mol-ppm (e.g., from about 10 mol-ppm to about 1000 mol-ppm of total sulfur, or at least about 100 mol-ppm (e.g., from about 100 mol-ppm to about 1000 mol-ppm) of total sulfur.

Improvements in the stability of reforming/RWGS catalysts described herein, particularly with respect to gaseous feed mixtures comprising non-CH₄ hydrocarbons and/or sulfur-bearing components as described herein that generally promote catalyst deactivation, may be attributed at least in part to their high activity, which manifests in lower operating (reactor or catalyst bed) temperatures. This, in turn, contributes to a reduced rate of the formation and deposition of coke on the catalyst surface and an extended, stable operation. In view of the ability of reforming/RWGS catalysts described herein to achieve a given or targeted level of performance (e.g., in terms of CH₄ conversion) at a relatively low operating (or average catalyst bed) temperature as a reforming/RWGS condition, such catalysts may alternatively be referred to as “cool” reforming catalysts, with the associated processes being referred to as “cool” reforming processes.

Representative reforming/RWGS catalysts suitable for catalyzing the reforming and/or RWGS reactions described herein comprise a noble metal, and possibly two, or even more than two, noble metals, on a solid support. The solid support may comprise cerium oxide, or, more particularly, cerium oxide in combination with a suitable binder (e.g., alumina) in a suitable amount (e.g., from about 5 wt-% to about 35 wt-%) to impart mechanical strength.

The phrase “on a solid support” is intended to encompass catalysts in which the active metal(s) is/are on the support surface and/or within a porous internal structure of the support. The solid support preferably comprises a metal oxide, with cerium oxide being of particular interest. Cerium oxide may be present in an amount of at least about 60 wt-% and preferably at least about 75 wt-%, based on the weight of the solid support (e.g., relative to the total amount(s) of metal oxide(s) in the solid support). Whether or not in oxide form, cerium may be present in an amount from about 30 wt-% to about 80 wt-%, and preferably from about 40 wt-% to about 65 wt-%, of the catalyst. The solid support may comprise all or substantially all (e.g., greater than about 95 wt-%) cerium oxide, or otherwise all or substantially all (e.g., greater than about 95 wt-%) of a combined amount of cerium oxide and a second metal oxide (e.g., aluminum oxide) that acts as a binder. One or more of other metal oxides, such as aluminum oxide, silicon oxide, titanium oxide, zirconium oxide, magnesium oxide, calcium oxide, iron oxide, vanadium oxide, chromium oxide, nickel oxide, tungsten oxide, strontium oxide, etc., may also be present, independently in individual amounts, or otherwise in combined amounts in the case of two or more of such other metal oxides, representing a minor portion, such as less than about 50 wt-%, less than about 30 wt-%, less than about 10 wt-%, or less than about 5 wt-%, of the solid support. Preferably, one or more of silicon oxide, titanium oxide, zirconium oxide, magnesium oxide, calcium oxide, iron oxide, vanadium oxide, chromium oxide, nickel oxide, tungsten oxide, and strontium oxide is substantially absent in the solid support. For example, these metal oxides may be present, independently in individual amounts, or otherwise in combined amounts in the case of two or more of such other metal oxides, of less than about 3 wt-%, less than about 0.5 wt-%, or even less than about 0.1 wt-%, of the solid support. For illustrative purposes, in specific embodiments, (i) silicon oxide (silica) may be present in an amount of less than about 0.5 wt-% of the solid support, (ii) nickel oxide may be present in amount of less than about 0.5 wt-% of the solid support, or (iii) silicon oxide and nickel oxide may be present in a combined amount of less than about 0.5 wt-% of the solid support. In other embodiments, the solid support may comprise one or more of such other metal oxides, including aluminum oxide, independently in individual amounts, or otherwise in combined amounts in the case of two or more of such other metal oxides, representing a major portion, such as greater than about 50 wt-%, greater than about 70 wt-%, or greater than about 90 wt-%, of the solid support. In such cases, the solid support may also optionally comprise cerium oxide in an amount representing a minor portion, such as less than about 50 wt-%, less than about 30 wt-%, or less than about 10 wt-%, of the solid support. Such minor portion of cerium oxide may also represent all or substantially all of the balance of the solid support, which is not represented by the one or more of such other metal oxides.

According to particular embodiments, the solid support may comprise, in addition to cerium oxide, a second metal oxide that acts as a binder for cerium oxide. Such second metal oxide may be selected from the group of other metal oxides described above, namely, aluminum oxide, silicon oxide, titanium oxide, zirconium oxide, magnesium oxide, calcium oxide, iron oxide, vanadium oxide, chromium oxide, nickel oxide, tungsten oxide, and strontium oxide. Such second metal oxide may be present in an amount generally from about 1 wt-% to about 45 wt-%, typically from about 5 wt-% to about 35 wt-%, and often from about 10 wt-% to about 25 wt-%, of the solid support. Preferably, the solid support comprises cerium oxide and the second metal oxide in a combined amount of generally at least about 85 wt-%, typically at least about 95 wt-%, and often at least about 99 wt-%, of the solid support. The solid support may comprise cerium oxide and the second metal oxide in a combined amount of generally at least about 85 wt-%, typically at least about 92 wt-%, and often at least about 95 wt-%, of the reforming/RWGS catalyst. A preferred second metal oxide that acts as a binder for cerium oxide is aluminum oxide.

A preferred property of the solid support (e.g., comprising predominantly cerium oxide), and consequently the reforming/RWGS catalyst, is low acidity. In this regard, excessive acid sites on the support or catalyst, and in particular strong, Brønsted acid sites, are believed to contribute to coking and catalyst deactivation during the reforming and/or RWGS reactions. Importantly, advantages of a low Brønsted acid site proportion, or concentration, in terms of establishing a commercially feasible catalyst life, are gained despite the fact that strong acid sites are known to promote the activity of a number of significant commercial reactions. An extensively used method for acid site strength determination and quantification with respect to solid materials is temperature programmed desorption using ammonia as a molecular probe (NH₃-TPD).

According to this method, a sample of the solid material is prepared by degassing and activation at elevated temperature and in an inert environment, in order to remove water and other bound species. The sample is then saturated with NH₃, with the saturation temperature (e.g., 100° C.) and subsequent purge with an inert gas (e.g., helium) providing conditions that remove any physisorbed NH₃. Temperature programmed desorption of the activated and saturated sample is initiated by ramping the temperature at a predetermined rate (e.g., 10° C./minute) to a final temperature (e.g., 400° C.) under the flow of the inert gas. The concentration of NH₃ in this gas is continually measured as it is driven from acid sites of the solid material having increasing strengths that correspond to increasing desorption temperatures. The determination of NH₃ concentration in the flowing inert gas can be performed, for example, using gas chromatography with a thermal conductivity detector (GC-TCD).

Typically, the NH₃ concentration versus temperature profile will include peaks at low and high temperatures that correspond to sites of the solid material having comparatively low and high acid strengths, respectively. The areas under these peaks can then provide relative concentrations of acids sites of the differing types of acid strength (e.g., expressed as a percentage of total acid sites), or otherwise these areas can be used to determine the absolute concentrations of the differing types (e.g., expressed in terms of milliequivalents per gram of the solid material). In the case of a solid support or reforming/RWGS catalyst that generates two peaks on the NH₃ concentration versus temperature profile over a relevant range, for example from 100° C. to 400° C., a first, low temperature peak may be associated with weak Lewis acid sites, whereas a second, high temperature peak may be associated with strong, Brønsted acid sites. For representative solid supports (e.g., comprising predominantly cerium oxide) as well as reforming/RWGS catalysts having such supports (in view of the relatively small or negligible impact, on the NH₃-TPD analysis, of catalytically active metals being deposited on such supports), the NH₃ concentration versus temperature profile obtained from an NH₃-TPD analysis over a temperature range from 100° C. to 400° C. (with such profile having, for example, two identifiable peaks) may exhibit a maximum NH₃ concentration at a temperature of less than about 300° C. (e.g., from about 150° C. to about 300° C.), and more typically at a temperature of less than about 250° C. (e.g., from about 150° C. to about 250° C.). This maximum NH₃ concentration may therefore be associated with a low temperature peak corresponding to weak Lewis acid sites, with the maximum NH₃ concentration and temperature at which this concentration is exhibited defining a point on this low temperature peak. Based on a peak area of this low temperature peak, relative to a peak area of a higher temperature peak corresponding to strong, Brønsted acid sites, the Lewis acid sites may represent at least about 25%, at least about 30%, or at least about 35%, of the total acid sites (e.g., the total Lewis and Brønsted acid sites combined). The higher temperature peak may, for example, exhibit a maximum NH₃ concentration at a temperature from about 300° C. to about 350° C., or, more typically, from about 300° C. to about 325° C. The maximum NH₃ concentration associated with the low temperature peak is normally greater than the maximum NH₃ concentration associated with the higher temperature peak, as a further indication that weak Lewis acid sites contribute to a substantial proportion of the overall acid sites of the solid support or reforming/RWGS catalyst. In representative embodiments, the solid support or reforming/RWGS catalyst may have a Lewis acid site concentration of at least about 0.25 milliequivalents per gram (meq/g) (e.g., from about 0.25 meq/g to about 1.5 meq/g), and more typically at least about 0.35 milliequivalents per gram (meq/g) (e.g., from about 0.35 meq/g to about 0.85 meq/g).

The solid support (e.g., comprising predominantly cerium oxide), as well as the reforming/RWGS catalyst comprising such support, may have a surface area from about 1 m²/g to about 100 m²/g, such as from about 10 m²/g to about 50 m²/g. Surface area may be determined according to the BET (Brunauer, Emmett and Teller) method based on nitrogen adsorption (ASTM D1993-03(2008)). The support and/or catalyst may have a total pore volume, of pores in a size range of 1.7-300 nanometers (nm), from about 0.01 cc/g to about 0.5 cc/g, such as from about 0.08 cc/g to about 0.25 cc/g. Pore volume may be measured by mercury porosimetry. The support and/or catalyst may have an average pore diameter from about 2 to about 75 nm, such as from about 5 to about 50 nm. The support and/or catalyst may have (i) from about 10% to about 80%, such as from about 30% to about 55%, of its pore volume attributed to macropores of >50 nm, (ii) from about 20% to about 85%, such as from about 35% to about 60%, of its pore volume attributed to mesopores of 2-50 nm, and/or (iii) less than about 2%, such as less than about 0.5%, of its pore volume attributed to micropores of <2 nm. Pore size distribution may be obtained using the Barrett, Joyner, and Halenda method.

Noble metals are understood as referring to a class of metallic elements that are resistant to oxidation. In representative embodiments, the noble metal, for example at least two noble metals, of the reforming/RWGS catalyst may be selected from the group consisting of platinum (Pt), rhodium (Rh), ruthenium (Ru), palladium (Pd), silver (Ag), osmium (Os), iridium (Ir), and gold (Au), with the term “consisting of” being used merely to denote group members, according to a specific embodiment, from which the noble metal(s) are selected, but not to preclude the addition of other noble metals and/or other metals generally. Accordingly, a catalyst comprising a noble metal embraces a catalyst comprising at least two noble metals, as well as a catalyst comprising at least three noble metals, and likewise a catalyst comprising two noble metals and a third, non-noble metal such as a promoter metal (e.g., a transition metal). According to preferred embodiments, the noble metal is present in an amount, or alternatively the at least two noble metals are each independently present in amounts, from about 0.05 wt-% to about 5 wt-%, from about 0.3 wt-% to about 3 wt-%, or from about 0.5 wt-% to about 2 wt-%, based on the weight of the catalyst. For example, a representative reforming/RWGS catalyst may comprise the two noble metals Pt and Rh, and the Pt and Rh may independently be present in an amount within any of these ranges (e.g., from about 0.05 wt-% to about 5 wt-%). That is, either the Pt may be present in such an amount, the Rh may be present in such an amount, or both Pt and Rh may be present in such amounts. A particularly preferred, noble metal-containing reforming/RWGS catalyst comprises both Pt and Rh, each independently present in an amount from about 0.5 wt-% to about 2 wt-%, on a support comprising, comprising substantially all, or consisting essentially of, cerium oxide and optionally a metal oxide binder (e.g., aluminum oxide) as described above. Regardless of the noble metal(s) used or the particular amounts used, preferably these noble metals are in their elemental (metallic or zero oxidation state) form. For example, with respect to the particularly preferred, noble metal-containing reforming/RWGS catalyst described above, such catalyst may comprise both Pt and Rh, each independently present in their respective elemental forms in an amount from about 0.5 wt-% to about 2 wt-%, based on the weight of the catalyst. Whereas other (compound) forms of Pt and/or Rh may also be present, preferably Pt and/or Rh in non-elemental forms, or noble metals generally in non-elemental forms, are present independently in individual amounts, or otherwise in combined amounts in the case of two or more noble metals, of less than about 1 wt-%, less than about 0.5 wt-%, or even less than about 0.1 wt-%, of the reforming/RWGS catalyst.

In representative embodiments, the at least two noble metals (e.g., Pt and Rh) may be substantially the only noble metals present in the reforming/RWGS catalyst, such that, for example, any other noble metal(s) is/are present in an amount or a combined amount of less than about 0.1 wt-%, or less than about 0.05 wt-%, based on the weight of the catalyst. In further representative embodiments, the at least two noble metals (e.g., Pt and Rh) are substantially the only metals present in the catalyst, with the exception of metals present in the solid support (e.g., such as cerium being present in the solid support as cerium oxide). For example, any other metal(s), besides at least two noble metals and metals of the solid support, may be present in an amount or a combined amount of less than about 0.1 wt-%, or less than about 0.05 wt-%, based on the weight of the catalyst. In some embodiments, certain metals may be substantially absent in the catalyst, whether in elemental form or in compound form (e.g., in the form of an oxide as a metal oxide component of the solid support). For example, certain metals may impart unwanted acidity in the solid support, provide insubstantial catalytic activity, and/or catalyze undesired reactions. In particular embodiments, one or more of Si, Ti, Zr, Mg, Ca, Fe, V, Cr, Ni, W, and Sr is substantially absent in the solid support. For example, these metals may be present, independently in individual amounts, or otherwise in combined amounts in the case of two or more of such metals, of less than about 0.5 wt-%, less than about 0.1 wt-%, or even less than about 0.05 wt-%, of the reforming/RWGS catalyst, or of the solid support for the catalyst. For example, one or more of Si, Zr, Mg, and Ni may be present in these individual amounts or combined amounts. Any metals present in the catalyst, including noble metal(s), may have a metal particle size in the range generally from about 0.3 nanometers (nm) to about 20 nm, typically from about 0.5 nm to about 10 nm, and often from about 1 nm to about 5 nm.

The noble metal(s) may be incorporated in the solid support according to known techniques for catalyst preparation, including sublimation, impregnation, or dry mixing. In the case of impregnation, which is a preferred technique, an impregnation solution of a soluble compound of one or more of the noble metals in a polar (aqueous) or non-polar (e.g., organic) solvent may be contacted with the solid support, preferably under an inert atmosphere. For example, this contacting may be carried out, preferably with stirring, in a surrounding atmosphere of nitrogen, argon, and/or helium, or otherwise in a non-inert atmosphere, such as air. The solvent may then be evaporated from the solid support, for example using heating, flowing gas, and/or vacuum conditions, leaving the dried, noble metal-impregnated support. The noble metal(s) may be impregnated in the solid support, such as in the case of two noble metals being impregnated simultaneously with both being dissolved in the same impregnation solution, or otherwise being impregnated separately using different impregnation solutions and contacting steps. In any event, the noble metal-impregnated support may be subjected to further preparation steps, such as washing with the solvent to remove excess noble metal(s) and impurities, further drying, calcination, etc. to provide the reforming/RWGS catalyst.

The solid support itself may be prepared according to known methods, such as extrusion to form cylindrical particles (extrudates) or oil dropping or spray drying to form spherical particles. Regardless of the specific shape of the solid support and resulting catalyst particles, the amounts of noble metal(s) being present in the catalyst, as described above, refer to the weight of such noble metal(s), on average, in a given catalyst particle (e.g., of any shape such as cylindrical or spherical), independent of the particular distribution of the noble metals within the particle. In this regard, it can be appreciated that different preparation methods can provide different distributions, such as deposition of the noble metal(s) primarily on or near the surface of the solid support or uniform distribution of the noble metal(s) throughout the solid support. In general, weight percentages described herein, being based on the weight of the solid support or otherwise based on the weight of catalyst, can refer to weight percentages in a single catalyst particle but more typically refer to average weight percentages over a large number of catalyst particles, such as the number in a catalyst bed within a reactor that is used in the a first or initial stage for carrying out reforming and/or RWGS.

### Reforming/RWGS Conditions

In the first (upstream) or initial stage, reforming and/or RWGS reactions, and preferably both simultaneously, are performed by contacting a gaseous feed mixture, preferably continuously using a flowing stream of the gaseous feed mixture to improve process efficiency, with reforming/RWGS catalyst as described herein. For example, contacting may be performed by continuously flowing the gaseous feed mixture through a reactor (which may be referred to as a reforming/RWGS reactor) that contains a noble metal-containing reforming/RWGS catalyst as described herein. The reactor maintains reforming/RWGS conditions, which are namely the conditions within a reactor vessel and, more particularly, within a bed of the reforming/RWGS catalyst that is contained in the vessel. These conditions include a temperature, pressure, and flow rate for the effective conversion of methane, and optionally other hydrocarbons, to hydrogen, in case such conditions are used to carry out reforming. Alternatively, but preferably in combination, these conditions are effective for the conversion of CO₂ to CO and thereby carry out the RWGS reaction.

Reforming/RWGS conditions that are useful for one or both of these reactions include a temperature generally from about 649° C. (1200° F.) to about 871° C. (1600° F.). In preferred embodiments, processes described herein, by virtue of the high activity of the catalyst, can effectively reform (oxidize) CH₄ and/or perform the RWGS reaction at significantly lower temperatures, compared to a representative conventional reforming temperature of 816° C. (1500° F.). For example, the reforming/RWGS conditions can include a temperature in a range from about 677° C. (1250° F.) to about 788° C. (1450° F.), or from about 704° C. (1300° F.) to about 760° C. (1400° F.). In the case of dry reforming that occurs if the gaseous feed mixture contains CO₂ as an oxidant for reforming, with relatively little or no H₂O and/or O₂, higher temperatures may be used, for example from about 843° C. (1550° F.) to about 1010° C. (1850° F.), or from about 885° C. (1625° F.) to about 941° C. (1725° F.). The presence of H₂S and/or other sulfur-bearing contaminants in significant concentrations (e.g., 100-1000 mol-ppm) may warrant increased temperatures, for example in a range from about 732° C. (1350° F.) to about 843° C. (1550° F.), or from about 760° C. (1400° F.) to about 816° C. (1500° F.), to maintain desired conversion levels (e.g., a CH₄ conversion of greater than about 85%). Advantageously, it has been discovered that the compensating effect of increasing temperature in response to increased sulfur concentrations in the gaseous feed mixture does not adversely affect catalyst stability. That is, the overall catalyst life is essentially unchanged, with respect to a comparison between a baseline sulfur-free operation and a sulfur-containing operation performed at a higher, compensating temperature.

Particularly in the case of large-scale operation, reactors operate with a limited release of heat to their surroundings (e.g., in the case of adiabatic operation), such that the catalyst bed temperature may vary as a given reaction proceeds (e.g., a fixed bed temperature profile may be characterized by an increasing or decreasing profile along the axial length of the reactor in the case of an exothermic or endothermic reaction, respectively). Accordingly, temperatures given herein that are associated with reforming/RWGS conditions, or otherwise downstream LPG synthesis reaction conditions, should be understood to mean average (or weighted average) catalyst bed temperatures. However, in view of the high activity of catalyst compositions described herein, particularly with respect to reforming/RWGS catalysts, temperatures given herein, and particularly those that are associated with reforming/RWGS conditions, in some embodiments may be maximum or peak catalyst bed temperatures.

Yet other reforming/RWGS conditions can include an above-ambient pressure, i.e., a pressure above a gauge pressure of 0 kPa (0 psig), corresponding to an absolute pressure of 101 kPa (14.7 psia). Because the reforming reactions make a greater number of moles of product versus moles of reactant, in some cases equilibrium may be favored at relatively low pressures. Representative reforming/RWGS conditions can include a gauge pressure generally from about 0 kPa (0 psig) to about 517 kPa (75 psig), typically from about 0 kPa (0 psig) to about 345 kPa (50 psig), and often from about 103 kPa (15 psig) to about 207 kPa (50 psig). According to some embodiments, it may be desirable to operate at higher pressures, for example in the range from about 207 kPa (30 psig) to about 6.9 MPa (1000 psig), from about 1.4 MPa (200 psig) to about 5.5 MPa (800 psig), or from about 2.1 MPa (300 psig) to about 4.8 MPa (700 psig). In some cases, it may be preferable that the pressure used in reactor(s) of the first stage (e.g., a reforming stage, such as a reforming/RWGS stage, or an RWGS stage) is the same or greater than the pressure used in reactor(s) of the second, LPG synthesis stage, such that an intervening operation of pressurization is avoided. Representative reforming/RWGS conditions may further include a WHSV generally from about 0.05 hr⁻¹ to about 10 hr⁻¹, typically from about 0.1 hr⁻¹ to about 8.0 hr⁻¹, and often from about 0.5 hr⁻¹ to about 5.0 hr⁻¹. As is understood in the art, the WHSV is the weight flow of the gaseous feed mixture (or total weight flow of all inputs to one or more reactors used in the reforming stage or RWGS stage) divided by the total weight of catalyst in the reforming/RWGS reactor(s) and represents the equivalent catalyst bed weights of the gaseous feed mixture (or all inputs) processed per hour. The WHSV is related to the inverse of the reactor residence time. The reforming/RWGS catalyst may be contained within the reactor(s) in the form of a fixed bed, but other catalyst systems are also possible, such as moving bed and fluidized bed systems that may be beneficial in processes using continuous catalyst regeneration. Regardless of the particular bed configuration, preferably the catalyst bed comprises discreet particles of reforming/RWGS catalyst, as opposed to a monolithic form of catalyst. For example, such discreet catalyst particles may have a spherical or cylindrical diameter of less than about 10 mm and often less than about 5 mm (e.g., about 2 mm). In the case of cylindrical catalyst particles (e.g., formed by extrusion), these may have a comparable length dimension (e.g., from about 1 mm to about 10 mm, such as about 5 min).

Advantageously, within any of the above temperature ranges and with respect to gaseous feed mixtures comprising CH₄, the high activity of the catalyst can achieve a conversion of this component of at least about 80% (e.g., from about 80% to about 99%), at least about 85% (e.g., from about 85% to about 99%), or at least about 90% (e.g., from about 90% to about 97%). A desired conversion level, with respect to a given gaseous feed mixture and reforming/RWGS catalyst, may be attained or controlled by adjusting the particular reactor or catalyst bed temperature and/or other reforming/RWGS conditions (e.g., WHSV and/or pressure) as would be appreciated by those having skill in the art, with knowledge gained from the present disclosure. Advantageously, noble metal-containing catalysts as described herein may be sufficiently active to achieve a significant CH₄ conversion, such as at least about 85%, in a stable manner at a temperature of at most about 732° C. (1350° F.), or even at most about 704° C. (1300° F.) (e.g., as a peak or maximum catalyst bed temperature). In the case of dry reforming, for example if the oxidant for reforming (according to the composition of the gaseous feed mixture) is predominantly, substantially all, or all CO₂ as described above, such CH₄ conversion levels may be achieved at higher temperatures, for example at most about 918° C. (1685° F.), or in some cases at most about to about 885° C. (1625° F.) (e.g., as a peak or maximum catalyst bed temperature). As is understood in the art, the conversion of CH₄ can be calculated on the basis of:

100*(CH4_{feed}-CH4_{prod})/CH4_{feed},

wherein CH4_{feed} is the total amount (e.g., total weight or total moles) of CH₄ in the gaseous feed mixture (or total amount in all inputs) provided to one or more reactors used in the reforming stage or RWGS stage and CH4_{prod} is the total amount of CH₄ in the synthesis gas intermediate obtained from this stage. In the case of continuous processes, these total amounts may be more conveniently expressed in terms of flow rates, or total amounts per unit time (e.g., total weight/hr or total moles/hr). These CH₄ conversion levels may be based on “per-pass” conversion, achieved in a single pass through a reforming/RWGS stage (e.g., a reforming/RWGS reactor of this stage), or otherwise based on overall conversion, achieved by returning a recycle portion of the LPG synthesis effluent back to the reforming/RWGS stage (e.g., a reforming/RWGS reactor of this stage), as described in greater detail below. In this regard, a recycle portion of an H₂/CO₂-enriched fraction of this effluent may also contain residual or unconverted CH₄ that can be converted in successive passes through the first reaction stage, thereby increasing CH₄ conversion on an overall basis.

In view of the CH₄ reforming reaction producing both H₂ and CO, the concentration of both of these components may be increased in the synthesis gas intermediate (product of reforming), relative to the gaseous feed mixture (or combined inputs to one or more reactors used in the reforming stage or RWGS stage). In some embodiments, depending on the H₂ concentration in the gaseous feed mixture and the extent of the RWGS reaction, the concentration of CO may be increased, whereas the concentration of H₂ may be decreased. In representative embodiments, the synthesis gas intermediate may comprise CO in an amount of at least about 5 mol-% (e.g., from about 5 mol-% to about 50 mol-%) or at least about 8 mol-% (e.g., from about 8 mol-%) to about 35 mol-%). In other embodiments, according to which high levels of conversion of CH₄ are achieved, the synthesis gas intermediate may comprise CO in a higher amount, such as at least about 30 mol-% (e.g., from about 30 mol-% to about 65 mol-%) or at least about 40 mol-% (e.g., from about 40 mol-% to about 55 mol-%). In further representative embodiments, the synthesis gas intermediate may comprise 1-12 in an amount of at least about 30 mol-% (e.g., from about 30 mol-% to about 90 mol-%) or at least about 40 mol-% (e.g., from about 40 mol-% to about 80 mol-%). With respect to the gaseous feed mixture, depending on the amount of H₂ present, as well as amounts of the oxidants CO₂ and H₂O present (which react with CH₄ to yield 1:1 and 3:1 stoichiometric molar ratios of H₂:CO, respectively) the H₂:CO molar ratio of the synthesis gas intermediate may be from about 1.0 to about 7.0, such as from about 4.0 to about 6.5, in the case of high ratios, Otherwise, in the case of lower ratios, the H₂:CO molar ratio of the synthesis gas intermediate may be from about 1.0 to about 3.0, such as from about 1.8 to about 2.4. According to yet other embodiments, for example in the case of CH₄ reforming with an oxidant that may be predominantly, substantially all, or all CO₂, the H₂:CO molar ratio of the synthesis gas intermediate may be less in view of the stoichiometry of the dry reforming reaction alone. For example, this H₂:CO molar ratio may be from about 0.5 to about 1.5, such as from about 0.8 to about 1.2.

In view of the reaction chemistry for subsequent LPG synthesis (e.g., via methanol synthesis and dehydration), the synthesis gas intermediate or portion thereof that is used for this step may have an H₂:CO molar ratio of at least 1.0 (e.g., from about 1.0 to about 3.5 or from about 1.5 to about 3.0), or more preferably at least about 2.0 (e.g., from about 2.0 to about 4.0, from about 2.0 to about 3.0, or from about 2.0 to about 2.5). In some cases, excess H₂ (i.e., H₂ in excess of the stoichiometric amount needed to react with CO and/or CO₂ to form a methanol intermediate according to the reactions below) may be desired to improve stability of a catalyst system used for the downstream LPG synthesis. In any event, molar ratios as described above may be representative of the synthesis gas intermediate or portion thereof used for LPG synthesis, as obtained directly from a reactor used in the reforming stage or the RWGS stage, or otherwise as obtained following an adjustment of the H₂:CO molar ratio, according to an intervening operation, for example by adding a source of H₂ and/or a source of CO to this intermediate or portion thereof, prior to (e.g., upstream of) the LPG synthesis stage. A representative source of H₂ and/or CO is an H₂/CO₂-enriched fraction, or a recycle portion thereof, of the LPG synthesis effluent, as described herein. Another representative source of H₂ and/or CO is hydrogen that has been purified (e.g., by PSA or membrane separation) or hydrogen that is impure (e.g., syngas). In other embodiments, between (a) the reforming stage or RWGS stage and (b) the LPG synthesis stage, water may be removed (e.g., condensed) from the synthesis gas intermediate or portion thereof used for LPG synthesis, for example to promote the dehydration (water formation) reaction.

### Liquefied Petroleum Gas (LPG) Synthesis

The first (upstream) or initial reaction stage, as described above, according to which a synthesis gas intermediate comprising both H₂ and CO (i.e., an H₂/C₀ mixture) is produced, may be followed by a second (downstream) stage of converting this synthesis gas intermediate or a portion thereof to propane and/or butane that is contained in the LPG product. This conversion of synthesis gas to LPG may proceed through a methanol synthesis reaction mechanism whereby, for example, methanol produced from H₂ and CO in the synthesis gas, according to a first pathway, is dehydrated to LPG hydrocarbons and water. In the case of producing propane (C₃H₈) and butane (C₄H₁₀) according to this reaction mechanism, the following, exemplary chemistry is illustrative:

14H₂+7CO→7CH₃OH and 7CH₃OH+2H₂→C₃H₈+C₄H₁₀+7H₂O.

Alternatively, but preferably in combination, CO₂ present in the synthesis gas intermediate or portion thereof that is used as a feed to the LPG synthesis stage (LPG synthesis feed) may likewise advantageously be reacted in the initial methanol synthesis, according to a second pathway. For example, in the case of producing the same number of moles of CH₃OH shown in reactions above that lead to the formation of propane and butane, CO₂, rather than CO, may be consumed according to:

21H₂+7CO₂→7CH₃OH+7H₂O.

With respect to the hydrogen requirement for methanol synthesis and dehydration according to the first pathway involving the hydrogenation of CO, the synthesis gas intermediate or portion thereof that is used in these steps may have an H₂:CO molar ratio as described above, or may be adjusted to obtain such H₂:CO molar ratio, to provide an LPG synthesis feed. In other embodiments, higher H₂:CO molar ratios of the LPG synthesis feed may be desirable, for example to account for the additional hydrogen consumption associated with the hydrogenation of CO₂ according to the second pathway. In general, representative processes may comprise feeding or inputting all or a portion of the synthesis gas intermediate, optionally following one or more intervening operations performed on this intermediate that may be used to provide an LPG synthesis feed having a composition and/or properties differing from that/those of the synthesis gas intermediate. Such intervening operations include cooling, heating, pressurizing, depressurizing, separation of one or more components (e.g., removal of condensed water), addition of one or more components (e.g., addition of H₂ and/or CO to adjust the molar H₂:CO ratio of an LPG synthesis feed relative to that of the synthesis gas intermediate), and/or reaction of one or more components (e.g., reaction of H₂ and/or CO using a separate water-gas shift reaction or reverse water-gas shift reaction), which operation(s) is/are performed on the synthesis gas intermediate to provide an LPG synthesis feed to LPG synthesis reactor(s) of an LPG synthesis stage.

In view of the temperatures and pressures typically used in the LPG synthesis reactor(s) of the LPG synthesis stage relative to those used in the reactor(s) of the reforming stage or RWGS stage, the synthesis gas intermediate may be cooled, separated from condensed water, and pressurized. In some embodiments, these may be the only intervening operations to which the synthesis gas intermediate is subjected, to provide an LPG synthesis feed. In other embodiments, cooling and pressurizing may be the only intervening operations. In yet other embodiments, an intervening operation may be the addition of (combination of the synthesis gas intermediate or portion thereof with) an H₂/CO₂-enriched fraction (or portion thereof, such as a recycle portion thereof) of an LPG synthesis effluent. This addition of an H₂/CO₂-enriched fraction or portion thereof may be the only intervening operation, or in some embodiments this may be combined with one or more of cooling, removal of condensed water, and pressurizing. In still other embodiments, intervening operations that may be omitted include drying of the synthesis gas intermediate to remove vapor phase H₂O (which is therefore different from condensing liquid phase H₂O and can include, e.g., using a sorbent selective for water vapor, such as 5A molecular sieve) and/or CO₂ removal according to conventional acid gas treating steps (e.g., amine scrubbing). According to some embodiments, CO₂ removal may be performed on the synthesis gas intermediate, upstream of the LPG synthesis stage (e.g., as an intervening operation). Preferably, prior to the LPG synthesis reactor(s), water produced in the reactor(s) of the reforming stage or RWGS stage is condensed from the synthesis gas intermediate, and/or also preferably the H₂:CO molar ratio of the synthesis gas intermediate is not adjusted. The use of no intervening operations between the reforming stage or RWGS stage and the LPG synthesis stage, limited intervening operations, and/or the omission or certain intervening operations, results in advantages associated with the overall simplification of processes for producing LPG products.

Conditions in the LPG synthesis stage, and more particularly LPG synthesis reactor(s) used in this stage, are suitable for the conversion of H₂ and CO to propane and/or butane of the LPG product. In representative embodiments, LPG synthesis reaction conditions, suitable for use in at least one LPG synthesis reactor or, more particularly, a catalyst bed contained in such reactor, can include an LPG synthesis reaction temperature in a range from about 204° C. (400° F.) to about 454° C. (850° F.), or from about 316° C. (600° F.) to about 399° C. (750° F.). As noted above, these temperatures may be understood as referring to average (or weighted average) catalyst bed temperatures, and alternatively, according to some embodiments may be maximum or peak catalyst bed temperatures. An LPG synthesis reaction pressure, suitable for use in at least one LPG synthesis reactor, can include a gauge pressure from about 690 kPa (100 psig) to about 6.9 MPa (1000 psig), such as from about 1.38 MPa (200 psig) to about 2.76 MPa (400 psig) or from about 3.4 MPa (500 psig) to about 5.2 MPa (750 psig).

### LPG Synthesis Catalyst Systems

In the LPG synthesis reactor(s), an LPG synthesis feed, representing all or a portion of the synthesis gas intermediate, optionally following one or more intervening operations described above, may be contacted with a suitable LPG synthesis catalyst (e.g., bed of LPG synthesis catalyst particles disposed within the LPG synthesis reactor) under LPG synthesis reaction conditions, which may include the temperatures and/or pressures as described above. Representative LPG synthesis catalysts may be considered “catalyst systems,” insofar as they may comprise at least two components having different catalytic activities, with such components either being (i) separate compositions (e.g., each composition being in the form of separate particles) of a methanol synthesis catalyst and a dehydration catalyst, or (ii) constituents of a bi-functional catalyst (e.g., the catalyst being in the form of separate particles) that is a single composition having both a methanol synthesis-functional constituent and a dehydration-functional constituent. In addition to such separate compositions of catalysts or single composition of a bi-functional catalyst, representative LPG synthesis catalyst systems may further comprise additional components, e.g., particles of silica or sand, acting to absorb heat and/or alter the distribution of solids. Such additional components may be present in an amount, for example, of at least 10 wt-%, at least 20 wt-%, or at least 40 wt-%, of a given catalyst system.

A representative methanol synthesis catalyst or methanol synthesis-functional constituent of a bi-functional catalyst may comprise one or more methanol synthesis-active metals, with representative metals being selected from the group consisting of copper (Cu), zinc (Zn), aluminum (Al), platinum (Pt), palladium (Pd), and chromium (Cr). These metals may be in their elemental forms or compound forms. For example, in the case of Cu, Pt, and Pd, these metals are preferably in their elemental forms and, in the case of Zn, Al, and Cr, these metals are preferably in their oxide forms, namely ZnO, Al₂O₃, and Cr₂O₃, respectively. In some preferred embodiments, all or a portion of Cu, in case of a methanol synthesis catalyst or methanol synthesis-functional constituent comprising this metal, may be in its oxide form CuO. A particular representative methanol synthesis catalyst is a copper and zinc oxide on alumina catalyst, comprising or consisting essentially of Cu/ZnO/Al₂O₃. Such “CZA” methanol synthesis catalyst may also be a methanol synthesis-functional constituent of a bi-functional catalyst.

For a methanol synthesis catalyst or a methanol synthesis-functional constituent comprising one or more of Cu, Zn, Al, Pt, Pd, and Cr, regardless of their particular form(s), such metal(s) may be present independently in an amount, in the respective methanol synthesis catalyst or bi-functional catalyst, generally from about 0.5 wt-% to about 45 wt-%, typically from about 1 wt-% to about 20 wt-%, and often from about 1 wt-% to about 10 wt-%, based on total catalyst weight. In some embodiments, the metal Cu may be present, in a methanol synthesis catalyst or bi-functional catalyst, in an amount from about 1 wt-% to about 25 wt-%, such as from about 1 wt-% to about 15 wt-%, based on total catalyst weight. Independently or in combination with such amounts of Cu, the metal Zn may be present, in a methanol synthesis catalyst or bi-functional catalyst, in an amount from about 1 wt-% to about 20 wt-%, such as from about 1 wt-% to about 10 wt-%, based on total catalyst weight. Independently or in combination with such amounts of Cu and/or Zn, the metal Al may be present, in a methanol synthesis catalyst or bi-functional catalyst, in an amount from about 1 wt-% to about 30 wt-%, such as from about 5 wt-% to about 20 wt-%, based on total catalyst weight. Independently or in combination with such amounts of Cu, Zn, and/or Al, any one or more of the metals Pt, Pd, and/or Cr may be present, in a methanol synthesis catalyst or bi-functional catalyst, independently in an amount, or in a combined amount, from about 1 wt-% to about 10 wt-%, such as from about 1 wt-% to about 5 wt-%, based on total catalyst weight.

In the case of a methanol synthesis catalyst or methanol synthesis-functional constituent of a bi-functional catalyst, the methanol synthesis-active metals Cu, Zn, Pt, Pd, and/or Cr, particularly when in their elemental forms, may be supported on a solid support. Representative solid supports comprise one or more metal oxides, selected from the group consisting of aluminum oxide, silicon oxide, titanium oxide, zirconium oxide, magnesium oxide, calcium oxide, strontium oxide, etc. The phrase “on a solid support” is intended to encompass methanol synthesis catalyst solid supports and bi-functional catalyst solid supports in which the methanol synthesis-active metal(s) is/are on the support surface and/or within a porous internal structure of the support.

In the case of a methanol synthesis catalyst or methanol synthesis-functional constituent of a bi-functional catalyst, the methanol synthesis-active metal(s), or any forms of such metals (e.g., their respective oxide forms), and optionally any solid support, may constitute all or substantially all of the catalyst or constituent. For example, the methanol synthesis-active metal(s), or any forms of such metals, and optionally any solid support, may be present in a combined amount representing at least about 90%, at least about 95%, or at least about 99%, of the total weight of the methanol synthesis catalyst or methanol synthesis-functional constituent.

A representative dehydration catalyst or dehydration-functional constituent of a bi-functional catalyst may comprise a zeolite (zeolitic molecular sieve) or a non-zeolitic molecular sieve. Particular zeolites may have a structure type selected from the group consisting of FAU, FER, MEL, MTW, MWW, MOR, BEA, LTL, MFI, LTA, EMT, ERI, MAZ, MEI, and TON, and preferably selected from one or more of FAU, FER, MWW, MOR, BEA, LTL, and MFI. The structures of zeolites having these and other structure types are described, and further references are provided, in Meier, W. M, et al., Atlas of Zeolite Structure Types, 4^{th} Ed Elsevier: Boston (1996). Specific examples include zeolite Y (FAU structure), zeolite X (FAU structure), MCM-22 (MWW structure), zeolite beta (BEA structure), and ZSM-5 (MFI structure), with zeolite beta and ZSM-5 being exemplary.

Non-zeolitic molecular sieves include ELAPO molecular sieves which are embraced by an empirical chemical composition, on an anhydrous basis, expressed by the formula:

(ELₓAl_{y}P_{z})O₂

wherein EL is an element selected from the group consisting of silicon, magnesium, zinc, iron, cobalt, nickel, manganese, chromium and mixtures thereof, x is the mole fraction of EL and is often at least 0.005, y is the mole fraction of aluminum and is at least 0.01, z is the mole fraction of phosphorous and is at least 0.01 and x+y+z=1. When EL is a mixture of metals, x represents the total mole fraction of such metals present. The preparation of various ELAPO molecular sieves is known, and examples of synthesis procedures and their end products may be found in U.S. Pat. No. 5,191,141 (ELAPO); U.S. Pat. No. 4,554,143 (FeAPO); U.S. Pat. No. 4,440,871 (SAPO); U.S. Pat. No. 4,853,197 (MAPO, MnAPO, ZnAPO, CoAPO); U.S. Pat. No. 4,793,984 (CAPO); U.S. Pat. Nos. 4,752,651 and 4,310,440. Preferred ELAPO molecular sieves are SAPO and ALPO molecular sieves. Generally, the ELAPO molecular sieves are synthesized by hydrothermal crystallization from a reaction mixture containing reactive sources of EL, aluminum, phosphorus and a templating agent. Reactive sources of EL are the metal salts of EL elements defined above, such as their chloride or nitrate salts. When EL is silicon, a preferred source is fumed, colloidal or precipitated silica. Preferred reactive sources of aluminum and phosphorus are pseudo-boehmite alumina and phosphoric acid. Preferred templating agents are amines and quaternary ammonium compounds. An especially preferred templating agent is tetraethylammonium hydroxide (TEAOH).

A particularly preferred dehydration catalyst or dehydration-functional constituent comprises an ELAPO molecular sieve in which EL is silicon, with such molecular sieve being referred to in the art as a SAPO (silicoaluminophosphate) molecular sieve. In addition to those described in U.S. Pat. Nos. 4,440,871 and 5,191,141, noted above, other SAPO molecular sieves that may be used are described in U.S. Pat. No. 5,126,308. Of the specific crystallographic structures described in U.S. Pat. No. 4,440,871, SAPO-34, i.e., structure type 34, represents a preferred component of an LPG synthesis catalyst system. The SAPO-34 structure is characterized in that it adsorbs xenon but does not adsorb iso-butane, indicating that it has a pore opening of about 4.2 Å. Accordingly, a representative dehydration catalyst or dehydration-functional constituent of a bi-functional catalyst may comprise SAPO-34 or other SAPO molecular sieve, such as SAPO-17, which is likewise disclosed in U.S. Pat. No. 4,440,871 and has a structure characterized in that it adsorbs oxygen, hexane, and water but does not adsorb iso-butane, indicative of a pore opening of greater than about 4.3 Å and less than about 5.0 Å. Due to its acidity, SAPO-34 can catalyze the conversion of a methanol intermediate to olefins such as propylene. Without being bound by theory, it is believed that the characteristic hydrogen partial pressures used in the LPG synthesis stage not only promote the hydrogenation of these olefins, but also stabilize the dehydration catalyst/functional constituent by preventing coking.

With respect to stability of catalysts as described herein, namely reforming/RWGS catalysts, methanol synthesis catalysts, dehydration catalysts, and bi-functional catalysts, it is believed that the presence of the byproduct formaldehyde may be detrimental in terms of its tendency to form coke precursors such as polycyclic aromatics. In this regard, further aspects of the invention relate to the use of yttrium in any of these catalysts, or as a separate component or catalyst composition. Without being bound by any particular theory as to advantages that may be gained from the use of yttrium, this metal is believed to have beneficial activity in terms of decomposing formaldehyde that may form/accumulate in one or both reaction stages of the process. Accordingly, in some embodiments, any of the catalyst compositions described herein may comprise, or further comprise, yttrium in elemental form or in compound form, such as in the form of yttria (yttrium oxide). For example, yttria may be used as a metal oxide component of the solid support for a reforming/RWGS catalyst as described above. With respect to any of the catalysts described herein, yttrium (e.g., in the form of yttria or other form) may be present in an amount from about 0.01 wt-% to about 10 wt-%, such as from about 0.05 wt-% to about 5 wt-% or from about 0.1 wt-% to about 1 wt-%. Otherwise, yttrium (e.g., in the form of yttria or other form) may be present as a separate composition, to provide a multi-composition reforming/RWGS catalyst system or LPG synthesis catalyst system having yttrium present in such amounts, in this case being relative to the total weight of a catalyst system having two or more separate compositions.

In a representative methanol synthesis catalyst or bi-functional catalyst, any metal(s) other than Cu, Zn, Al, Pt, Pd, and/or Cr may be present in minor amounts. For example, any such other metal(s) may be independently present in an amount of less than about 1 wt-%, less than about 0.1 wt-%, or even less than about 0.05 wt-%, based on the total catalyst weight. Alternatively, any two or more of such other metals may be present in a combined amount of less than about 2 wt-%, less than about 0.5 wt-%, or even less than about 0.1 wt-%, based on the total catalyst weight. According to particular embodiments, especially in the case of (i) a methanol synthesis catalyst comprising a solid support, or (ii) a bi-functional catalyst comprising, as a dehydration-functional constituent, a zeolite or non-zeolitic molecular sieve, such metals other than Cu, Zn, Al, Pt, Pd, and/or Cr, and present in the amounts described above, may be, more particularly, (i) metals other than Cu, Zn, Al, Pt, Pd, Cr, and Si; metals other than Cu, Zn, Al, Pt, Pd, Cr, Si, Ti, Zr, Mg, Ca, and Sr; or metals other than Cu, Zn, Al, Pt, Pd, Cr, Si, Ti, Zr, Mg, Ca, Sr, and Y, or (ii) metals other than Cu, Zn, Al, Pt, Pd, Cr, Si, and P; metals other than Cu, Zn, Al, Pt, Pd, Cr, Si, P, Mg, Zn, Fe, Co, Ni, and Mn; or metals other than Cu, Zn, Al, Pt, Pd, Cr, Si, P, Mg, Zn, Fe, Co, Ni, Mn, and Y.

In the case of a dehydration catalyst or dehydration-functional constituent of a bi-functional catalyst, a zeolite or non-zeolitic molecular sieve may constitute all or substantially all of the catalyst or constituent. For example, the zeolite or non-zeolitic molecular sieve, may be present in an amount representing at least about 90%, at least about 95%, or at least about 99%, of the total weight of the dehydration catalyst or dehydration-functional constituent. In the case of a bi-functional catalyst, the combined amount of (i) the methanol synthesis-active metal(s), or any forms of such metals (e.g., their respective oxide forms), and optionally any solid support, and (ii) a zeolite or non-zeolitic molecular sieve, may constitute all or substantially all of the bi-functional catalyst. For example, (i) and (ii) may be present in a combined amount representing at least about 90%, at least about 95%, or at least about 99%, of the total weight of the bi-functional catalyst.

A particular embodiment for carrying out LPG synthesis therefore involves the use of a single catalyst composition, namely a bi-functional catalyst comprising both a methanol synthesis-functional constituent and a dehydration-functional constituent, with these constituents corresponding in isolation to a methanol synthesis catalyst and a dehydration catalyst as described above. When combined in a single catalyst composition, the functional constituents of a bi-functional catalyst may be present in equal or substantially equal weight ratios. For example, the (i) methanol synthesis-functional constituent and (ii) dehydration-functional constituent may be present in the bi-functional catalyst in a weight ratio of (i):(ii) of about 1:1. Generally, however, this weight ratio may vary, for example the weight ratio of (i):(ii) may be from about 10:1 to about 1:10, such as from about 5:1 to about 1:5, or from about 3:1 to about 1:3. A representative bi-functional catalyst may therefore comprise (i) a methanol synthesis-functional constituent comprising one or more methanol synthesis-active metals as described above, and optionally a solid support as described above, and (ii) a dehydration-functional constituent comprising a zeolite or non-zeolitic molecular sieve as described above. It can be appreciated from the above description, including the weight ratios in which (i) and (ii) may be combined, that the one or more methanol synthesis-active metals may be present in a bi-functional catalyst as a whole, in an amount, or combined amounts, that is/are less than that/those amounts in which they are present in a methanol synthesis catalyst, as described above. Likewise, the zeolite or non-zeolitic molecular sieve may be present in a bi-functional catalyst as a whole, in an amount that is less than that in which it is present in a dehydration catalyst. For example, a bi-functional catalyst as a whole may comprise the one or more methanol synthesis-active metals in lower amount, such as in an amount generally from about 0.2 wt-% to about 30 wt-%, typically from about 0.5 wt-% to about 15 wt-%, and often from about 1 wt-% to about 5 wt-%, based on the weight of the bi-functional catalyst. Likewise, a bi-functional catalyst as a whole may comprise a zeolite or non-zeolitic molecular sieve in an amount from about 5 wt-% to about 90 wt-%, from about 10 wt-% to about 80 wt-%, or from about 35 wt-% to about 75 wt-%, based on the weight of the bi-functional catalyst.

The LPG synthesis catalysts and LPG synthesis reaction conditions described herein are generally suitable for achieving a conversion of H₂ and/or CO (H₂ conversion or CO conversion) of at least about 20% (e.g., from about 20% to about 99% or from about 20% to about 95%), at least about 30% (e.g., from about 30% to about 99% or from about 30% to about 95%), or at least about 50% (e.g., from about 50% to about 95% or from about 75% to about 95%). Whether these LPG synthesis conversion levels are based on H₂ conversion or CO conversion may depend on which reactant is stoichiometrically limited in the LPG synthesis feed, or in the synthesis gas intermediate, considering the LPG synthesis reaction chemistry. These LPG synthesis conversion levels may correspond to “per-pass” conversion levels, obtained in a single pass of the LPG synthesis feed through the LPG synthesis stage, or through a reactor in this stage. These conversion levels may be calculated in an analogous manner to that as described above, with respect to the conversion of CH₄ in a first reaction stage. Preferably, these LPG synthesis conversion levels are based on CO conversion, and more particularly based on conversion of CO in the synthesis gas intermediate or LPG synthesis feed (e.g., obtained following an intervening operation as described above). However, these LPG synthesis conversion levels may alternatively be based H₂ and/or CO that is input to the first reaction stage, i.e., that is present in the gaseous feed mixture or present in the fresh makeup feed. Another important performance parameter with respect to the LPG synthesis stage is carbon selectivity to LPG hydrocarbons, which refers to percentage of carbon (e.g., present in CO and CO₂) that is input to this stage and that manifests in LPG hydrocarbons, namely propane and/or butane (including both of the butane isomers, iso- and normal-butane) in the LPG synthesis effluent. In representative embodiments, carbon selectivity to LPG hydrocarbons is at least about 20% (e.g., from about 20% to about 90% or from about 20% to about 75%), at least about 30% (e.g., from about 30% to about 90% or from about 30% to about 75%), at least about 40% (e.g., from about 40% to about 90% or from about 40% to about 75%), or even at least about 50% (e.g., from about 50% to about 90% or from about 50% to about 75%). The carbon selectivity to propane may be at least about 10% (e.g., from about 10% to about 60% or from about 10% to about 50%), at least about 15% (e.g., from about 15% to about 60% or from about 15% to about 50%), or at least about 20% (e.g., from about 20% to about 60% or from about 20% to about 50%). The carbon selectivity to butane (both iso- and normal-butane) may be at least about 5% (e.g., from about 5% to about 45% or from about 5% to about 35%), at least about 10% (e.g., from about 10% to about 45% or from about 10% to about 35%), or at least about 15% (e.g., from about 15% to about 45% or from about 15% to about 35%). Preferably, these carbon selectivity levels are based on the total carbon present (e.g., as CO and CO₂) in the synthesis gas intermediate or LPG synthesis feed (e.g., obtained following an intervening operation as described above). However, these carbon selectivity levels may alternatively be based on the total carbon that is input (e.g., as CO, CO₂, and CH₄) to the first reaction stage, i.e., that is present in the gaseous feed mixture or present in the fresh makeup feed.

A per-pass (or single pass) yield of LPG hydrocarbons provides a further, important measure of performance of the LPG synthesis stage. This per-pass yield refers to the product of the per-pass CO conversion and the carbon selectivity to LPG hydrocarbons. In representative processes, the per-pass yield of LPG hydrocarbons (or LPG hydrocarbon yield) is at least about 15% (e.g., from about 15% to about 85% or from about 15% to about 70%), at least about 25% (e.g., from about 25% to about 85% or from about 25% to about 70%), at least about 35% (e.g., from about 35% to about 85% or from about 35% to about 70%), or even at least about 45% (e.g., from about 45% to about 85% or from about 45% to about 70%). In some preferred embodiments, the per-pass yield of LPG hydrocarbons in the LPG synthesis stage is at least about 50%.

A desired H₂ conversion and/or CO conversion in the LPG synthesis reactor(s), as well as other desired performance parameters, may be achieved by adjusting the LPG synthesis reaction conditions described above (e.g., LPG synthesis reaction temperature and/or LPG synthesis reaction pressure), and/or adjusting the weight hourly space velocity (WHSV). The LPG synthesis reaction conditions may include a weight hourly space velocity (WHSV) generally from about 0.01 hr⁻¹ to about 10 hr⁻¹, typically from about 0.05 hr⁻¹ to about 5 hr⁻¹, and often from about 0.1 hr⁻¹ to about 1.5 hr⁻¹, as defined above and based on the combined weight of the methanol synthesis catalyst and dehydration catalyst, or otherwise based on the weight of the bi-functional catalyst, as described above. The conversion level (e.g., CO conversion) may be increased, for example, by increasing pressure and decreasing WHSV, having the effects, respectively, of increasing reactant concentrations and reactor residence times.

Embodiments of the invention are therefore directed to a process for producing an LPG product from a synthesis gas comprising H₂ and CO, for example a synthesis gas intermediate or an LPG synthesis feed obtained following one or more intervening operations performed on this intermediate, as described above. More broadly, any source of synthesis gas may be used as an LPG synthesis feed in representative LPG synthesis processes, including LPG synthesis feeds having an H₂:CO molar ratio that is representative of a synthesis gas intermediate, as described above. The synthesis gas intermediate or LPG synthesis feed may generally be produced by reforming and/or RWGS reactions, as described above. However, with respect to LPG synthesis processes do not require a specific source of synthesis gas, representative embodiments are directed to such processes (e.g., single stage LPG synthesis processes) that do not necessarily require a given upstream conversion step (e.g., a reforming stage as described herein). Representative processes comprise contacting broadly any source synthesis gas, or more specifically any particular synthesis gas intermediate or LPG synthesis feed as described herein, with an LPG synthesis catalyst system as described herein, such as mixture of (i) a methanol synthesis catalyst and (ii) a dehydration catalyst, as described above, for example wherein (i) may comprise, or consist essentially of, one or more methanol synthesis-active metals selected from Cu, Zn, Al, Pt, Pd, and/or Cr and optionally a solid support, as described above, and (ii) may comprise, or consist essentially of, a zeolite or a non-zeolitic molecular sieve as described above. The process comprises converting H₂ and CO, and optionally CO₂, in the synthesis gas to hydrocarbons, including propane and/or butane that are provided in the LPG product. Other particular embodiments are directed to a process for producing an LPG product comprising propane and/or butane, comprising (a) in a reforming stage or an RWGS stage, contacting a gaseous feed mixture (e.g., in the case of a recycle operation, contacting a gaseous feed mixture comprising both a fresh makeup feed and a recycle portion of an H₂/CO₂-enriched fraction) with a reforming/RWGS catalyst to produce a synthesis gas intermediate comprising an H₂/CO mixture. The gaseous feed mixture may, for example, comprise CH₄, CO₂, and H₂ in a combined amount of at least 30 mol-%. The process may further comprise (b) in an LPG synthesis stage, contacting at least a portion of the synthesis gas intermediate with an LPG catalyst system as described herein, to produce an LPG synthesis effluent.

The LPG product comprising propane and/or butane may therefore be obtained following a step of converting a synthesis gas intermediate via LPG synthesis. The LPG product may correspond to the LPG synthesis effluent of an LPG synthesis reactor (e.g., the LPG product may be obtained without further processing of the LPG synthesis effluent) or otherwise the LPG product may be separated from the LPG synthesis effluent, for example as a fraction of the LPG synthesis effluent enriched in propane and/or butane that is separated using techniques known in the art (e.g., fractionation). In either case, the LPG synthesis effluent may be obtained directly from the LPG synthesis stage (e.g., an LPG synthesis reactor of this stage). In preferred embodiments, therefore, processes described herein comprise a step, following the two reaction stages, of separating the LPG product from the LPG synthesis effluent. In addition to this LPG product, processes may further comprise separating one or more other fractions from the LPG synthesis effluent, such as fractions that are depleted in LPG hydrocarbons, relative to the LPG product. For example, such other fraction(s) may include an H₂/CO₂-enriched fraction, i.e., a fraction that is enriched in H₂ and CO, relative to the LPG synthesis effluent and the LPG product. Such other fraction(s) may, alternatively or in combination, include a water-enriched fraction, i.e., a fraction that is enriched in water, relative to the LPG synthesis effluent and the LPG product. Both such H₂/CO₂-enriched fraction and water-enriched fraction represent fractions that, following their separation from the LPG synthesis effluent, may advantageously be recycled in the process, as described in greater detail below. The H₂/CO₂-enriched fraction and water-enriched fractions may, respectively, represent gaseous (vapor) and liquid fractions separated from the LPG synthesis effluent, e.g., as respective, lower-boiling (more volatile) and higher-boiling (less volatile) fractions, relative to the LPG product.

According to specific embodiments, the LPG product (e.g., following separation) may comprise propane and butane in a combined amount of at least about 60 mol-% (e.g., from about 60 mol-% to about 100 mol-%), at least about 80 mol-% (e.g., from about 80 mol-% to about 100 mol-%), or at least about 90 mol-% (e.g., from about 90 mol-% to about 99 mol-%). Together with such combined amounts, or alternatively, the LPG product may comprise propane and/or butane independently in individual amounts of at least about 25 mol-% (e.g., from about 25 mol-% to about 85 mol-%), at least about 40 mol-% (e.g., from about 40 mol-% to about 80 mol-%), or at least about 50 mol-% (e.g., from about 50 mol-% to about 75 mol-%). The balance of the LPG product may comprise all, or substantially all, pentane or a combination of ethane and pentane. According to other specific embodiments, at least about 40% (e.g., from about 40% to about 95%), at least about 55% (e.g., from about 55% to about 95%), or at least about 70% (e.g., from about 70% to about 95%) of the carbon content of the gaseous feed mixture (e.g., the carbon content of CH₄ and/or CO₂ present in this mixture), or alternatively the carbon content of the fresh makeup feed, forms propane and/or butane of the LPG product. These percentages are equivalently expressed in terms of wt-% or mol-%.

### Once-Through and Recycle Operation/Exemplary Embodiment

Processes as described herein for producing an LPG product may be carried out with (configured for) once-through operation, whereby the gaseous feed mixture is input and the LPG product (optionally following separation from an LPG synthesis effluent, as described above) is withdrawn, without recycle of any portion of material obtained in the first or second reaction stages. In the case of once-through operation, the “gaseous feed mixture” and “fresh makeup feed” are normally equivalent, and the conversion levels and product yields obtained from the process represent those of a single pass through the stages of reforming and/or RWGS and LPG synthesis. As described above, certain aspects of the invention are associated with LPG production process that allow for the effective management/conversion of CO₂ that is present in a gaseous mixture or a fresh makeup feed, which can be improved through recycle operation. In particular, the recycle of CO₂ (e.g., present in an H₂/CO₂-enriched fraction that may be separated from an LPG synthesis effluent), back to the first stage (e.g., a reforming stage, such as a reforming/RWGS stage), and/or back to the second, LPG synthesis stage for further reaction, can promote its complete or essentially complete, overall conversion. For example, in representative embodiments in which recycle operation is used as described herein, an overall conversion of CO₂ present in a fresh makeup feed (e.g., having a composition as described above with respect to a “gaseous feed mixture”) may be at least about 90%, at least about 95%, or even at least about 99%, with deviations from complete or 100% conversion resulting substantially, or at least in part, from CO₂ losses in a purge exiting the gaseous recycle loop that is used to control the accumulation of unwanted impurities in this loop. That is, according to some embodiments, CO₂ introduced to the process in the gaseous feed mixture or fresh makeup feed may be recycled substantially to extinction. In terms of fractions, as described above, which may be separated and/or recovered from the LPG synthesis effluent, an H₂/CO₂-enriched fraction and/or a water-enriched fraction may, for example, be recycled to the first stage (e.g., a reforming stage, such as a reforming/RWGS stage) and/or to the second, LPG synthesis stage to attain important advantages as described herein. In some cases, only an H₂/CO₂-enriched fraction, or a recycle portion thereof, is recycled. For example, a recycle portion of the H₂/CO₂-enriched fraction may be recycled to the second stage, or otherwise parts of this recycle portion may be recycled to the first and second stages.

An exemplary embodiment of a process 1 for producing an LPG product and utilizing recycle is depicted in the FIGURE. As illustrated, gaseous feed mixture 6 is provided to reforming stage or RWGS stage 100, which may include one or more reforming/RWGS reactors for contacting gaseous feed mixture 6 with a reforming/RWGS catalyst and under reforming/RWGS conditions as described herein. Reactions occurring in reforming stage or RWGS stage 100 produce synthesis gas intermediate 8, which may be subjected to any one or more intervening operations as described herein. For example, water, such as in the form of condensed liquid water 9, may be separated from synthesis gas intermediate 8 to provide LPG synthesis feed 10. Optionally or in combination with this removal of condensed liquid water 9, a portion of H₂/CO₂-enriched fraction 14 of LPG synthesis effluent 12 may be added to synthesis gas intermediate 8 to provide LPG synthesis feed 10. For example, second part 4b of this fraction may be added as illustrated, having the effect of altering the composition of LPG synthesis feed 10, relative to that of synthesis gas intermediate 8, and more particularly with respect to the H₂:CO ratio of LPG synthesis feed. Whether or not any intervening operations are performed, LPG synthesis feed 10 (which in the absence of any intervening operation will be the same as synthesis gas intermediate 8), or a portion thereof, is provided to LPG synthesis stage 200, which may include one or more LPG synthesis reactors for contacting LPG synthesis feed 10 (or synthesis gas intermediate 8) with an LPG synthesis catalyst system and under LPG synthesis conditions as described herein. Reactions occurring in LPG synthesis stage 200 produce LPG synthesis effluent 12 that may be obtained directly from LPG synthesis stage 200. All or a portion of LPG synthesis effluent 12, optionally following a further intervening operation such as cooling via cooler 250, may be provided to separation stage 300 for separating various fractions as described above. According to the particular embodiment illustrated in the FIGURE, the separated fractions may include (e.g., among one or more other fractions), or may consist of, H₂/CO₂-enriched fraction 14 and water-enriched fraction 18, in addition to LPG product 16 comprising LPG hydrocarbons as described herein. Relative to other fractions 14, 18 separated from LPG synthesis effluent 12, LPG product 16 is enriched in both propane and butane (based on a combined amount of iso- and normal-butane), and in preferred embodiments has amounts of propane and/or butane as described above.

To improve overall CO₂ conversion and management, at least a portion of H₂/CO₂-enriched fraction 14 may be recycled back to upstream operations or stages of the process, including reforming stage or RWGS stage 100, and/or LPG synthesis stage 200. Typically, for example, a recycle portion 4 of H₂/CO₂-enriched fraction may be obtained following the removal of purge 20 that serves to limit the accumulation of unwanted impurities in the gaseous recycle loop, and particularly non-condensable impurities such as N₂ and others that may be present in fresh makeup feed 2. The separation of purge 20 provides recycle portion 4 of the H₂/CO₂-enriched fraction 14, which recycle portion 4 may then, using recycle gas compressor 350, be advantageously utilized to improve performance of the overall process in various respects. For example, recycle portion 4 may be recycled to either or both stages 100, 200 to increase overall CO₂ conversion of the process (e.g., beyond a “per-pass” or once-through CO₂ conversion that may be obtained on the basis of either stage operating alone, or on the basis of both stages operating together). Alternatively, or in combination, CO₂ present in H₂/CO₂-enriched fraction 14 or recycle portion 4 thereof, may, when introduced to one or both stages 100, 200, and particularly LPG synthesis stage 200, beneficially suppress or reduce a net CO₂ production in that stage (e.g., due to the water-gas shift reaction). According to the particular embodiment shown in the FIGURE, a first part 4a of recycle portion 4 may be recycled to reforming stage or RWGS stage 100 (e.g., by being combined with fresh makeup feed 2), and/or a second part 4b may be recycled to LPG synthesis stage 200 (e.g., by being combined with synthesis gas intermediate 8 or LPG synthesis feed 10). The selection of a given recycle configuration, in terms of recycling H₂/CO₂-enriched fraction 14 or any portion(s) thereof to certain stage(s) of the process, may depend at least in part on the above considerations with respect to increasing overall CO₂ conversion of the process and/or suppressing CO₂ production in a given stage. Having knowledge of the present disclosure, the skilled person would appreciate the applicability of these and other considerations to a given process within the scope of invention. As is apparent from the above description, the recycle portion 4 as well as any parts 4a, 4b thereof that may be routed to different locations all constitute “a portion of the H₂/CO₂-enriched fraction 14,” for purposes of the present disclosure. Therefore, for example, gaseous feed mixture 6 may be provided to reforming stage or RWGS stage 100 as a combination of fresh makeup feed 2 and a portion of the H₂/CO₂-enriched fraction 14 (e.g., all of recycle portion 4, or part 4a of this portion), optionally further in combination with water-enriched fraction 18, which may be recycled using recycle liquid pump 450.

According to particular embodiments, fresh makeup feed 2 may comprise, or consist essentially of, biogas. In such embodiments, gaseous feed mixture 6 may comprise biogas that is present therein as a fresh makeup feed portion.

### Examples

The following examples are set forth as representative of the present invention. These examples are not to be construed as limiting the scope of the invention as other equivalent embodiments will be apparent in view of the present disclosure and appended claims.

An LPG synthesis catalyst system of 1 gram of methanol synthesis catalyst (Cu/ZnO/Al₂O₃), 3 grams of zeolite beta, and 1 gram of sand was tested for its activity to convert a 2:1 H₂:CO molar ratio synthesis gas. In separate tests of Examples 1-3, normal flow rates of the synthesis gas in ml/min of 165, 110, and 55 were used, respectively, in conjunction with other LPG synthesis conditions of 2.1 MPa (300 psig) gauge pressure and 350° C. (662° F.) catalyst bed temperature. Results are summarized in Table 1 below, including CO conversion and percent carbon selectivity for various components of the LPG synthesis effluent.

TABLE 1
Variations in LPG synthesis feed flow rate

| Example | 1 | 2 | 3 |
|---|---|---|---|
| Flow rate, ml/min | 165 | 110 | 55 |
| WHSV, hr⁻¹ (based on 4 g catalyst) | 1.1 | 0.71 | 0.35 |
| CO Conversion | 83.5% | 88.0% | 91.4% |
| | Carbon Selectivity | | |
| CH₄ | 6% | 3% | 4% |
| CO₂ | 45% | 41% | 39% |
| ethane | 7% | 9% | 9% |
| propane | 22.4% | 27.9% | 29.8% |
| i-butane | 12.4% | 11.6% | 10.4% |
| n-butane | 5.5% | 6.7% | 7.0% |
| i-pentane | 1% | 1% | 1% |
| n-pentane | 0.0% | 0.0% | 0.0% |
| 2-methyl-pentane | 0.1% | 0.1% | 0.1% |
| 3-methyl-pentane | 0.0% | 0.1% | 0.0% |
| methanol | 0.0% | 0.1% | 0.0% |
| LPG hydrocarbons | 40% | 46% | 47% |
| C3 fraction of LPG | 0.56 | 0.60 | 0.63 |
| LPG Yield | 34% | 40% | 43% |

As is apparent from these results, the exemplary LPG synthesis catalyst system was active under the conditions described above, for converting synthesis gas to LPG hydrocarbons (propane and the iso- and normal-butane isomers) with a favorable CO conversion in a range of about 83-92% and carbon selectivity in a range of about 40-48%. Whereas it is believed that a methanol synthesis and dehydration reaction mechanism accounted for the production of these and other hydrocarbons, it is evident that the methanol intermediate was present in the LPG synthesis effluent in only trace or undetectable quantities. In addition, these results illustrate the impact of reducing the rate of the synthesis gas used as a representative LPG synthesis feed. In particular, lowering the feed rate had the effect of increasing CO conversion, at least in part due to the increase in reactor residence time (decrease in WHSV). As would be understood by those skilled in the art having knowledge of the present disclosure, the feed rate and other LPG synthesis conditions can be varied to achieve other ranges of conversion levels.

The experiment described above in Example 1 and performed with a normal flow rate of the synthesis gas of 165 ml/min, was used as a baseline experiment for comparison purposes. Specifically, the 2:1 H₂:CO molar ratio synthesis gas, i.e., BASELINE FEED having an approximate H₂/CO composition of 67 mol-%/33 mol-%, used in this experiment as an LPG synthesis feed, was varied in subsequent experiments, in terms of its composition, to evaluate differences in performance that could be obtained with other LPG synthesis feeds. These feeds had compositions of:
(A) (i) 50 mol-% of 2:1 H₂:CO molar ratio synthesis gas, combined with (ii) 50 mol-% CO₂—FEED A, having an approximate H₂/CO₂/C₀ composition of 33.5 mol-%/50 mol-%/16.5 mol-% (Example 4);
(B) a 3:1 H₂:CO molar ratio synthesis gas—FEED B, having an approximate H₂/CO composition of 75 mol-%/25 mol-% (Example 5); and
(C) (i) 2:1 H₂:CO molar ratio synthesis gas, combined with (ii) an H₂/CO₂-enriched fraction of an LPG synthesis effluent and representative of an LPG synthesis feed obtained from recycle operation as described herein—FEED C, having an approximate H₂/CO₂/C₀ composition of 64 mol-%/20.5 mol-%/15.5 mol-% (Example 6).

Therefore, compared to BASELINE FEED, as can be appreciated from the above description,

FEED A, FEED B, and FEED C were representative of comparative LPG synthesis feeds having, respectively, (i) an added amount of CO₂, (ii) an added amount of H₂, and (iii) added amounts of both H₂ and CO₂, as would be obtained from recycle operation as described herein. These LPG synthesis feeds were evaluated with respect to their conversion to LPG hydrocarbons and other components, under LPG synthesis conditions of 2.1 MPa (300 psi) gauge pressure and 350° C. (662° F.) catalyst bed temperature. These conditions were maintained in the presence of the exemplary LPG synthesis catalyst system of 1 gram of methanol synthesis catalyst (Cu/ZnO/Al₂O₃), 3 grams of zeolite beta, and 1 gram of sand, to carry out the LPG synthesis reaction. Results are summarized in Table 2 below, including CO conversion and percent carbon selectivity for various components of the LPG synthesis effluent.

TABLE 2
Variations in LPG synthesis feed composition

| | Composition, mol-% H₂/mol-% CO₂/mol-% CO | | | |
|---|---|---|---|---|
| | | FEED A, | FEED B, | FEED C, |
| | BASELINE, 67/0/33 | 33.5/50/16.5 | 75/0/25 | 64/20.5/15.5 |
| Example | 1 | 4 | 5 | 6 |
| Flow rate, ml/min | 165 | 165 | 165 | 165 |
| CO Conversion | 83.5% | 19.7% | 84.3% | 51.6% |
| | Carbon Selectivity | | | |
| CH₄ | 6% | 25% | 7% | 13% |
| CO₂ | 45% | N/A | 42% | 6% |
| ethane | 7% | 17% | 6% | 12% |
| propane | 22.4% | 32.6% | 25.0% | 40.0% |
| i-butane | 12.4% | 18.3% | 14.3% | 21.1% |
| n-butane | 5.5% | 6.5% | 5.1% | 6.9% |
| i-pentane | 1% | 1% | 1% | 1% |
| n-pentane | 0.0% | 0.0% | 0.5% | 0.0% |
| 2-methyl-pentane | 0.1% | 0.1% | 0.1% | 0.2% |
| 3-methyl-pentane | 0.0% | 0.1% | 0.0% | 0.1% |
| methanol | 0.0% | 0.0% | 0.0% | 0.0% |
| LPG hydrocarbons | 40% | 57% | 44% | 68% |
| C3 fraction of LPG | 0.56 | 0.57 | 0.56 | 0.59 |
| LPG Yield | 34% | 11% | 37% | 35% |

From the above results, it is evident that, compared to the BASELINE FEED, adding CO₂ alone to obtain FEED A (Example 4) caused a significant reduction in the rate of the LPG synthesis reaction and therefore the CO conversion. It is believed that this effect was due not only to the dilution of the CO reactant and corresponding decrease in its concentration or partial pressure in the reaction mixture, but also to a suppression by CO₂ of the LPG synthesis reaction. Therefore, in cases of LPG synthesis feeds being representative of significant CO₂ addition to synthesis gas, a substantial increase in catalyst, or otherwise a substantial decrease in feed rate (throughput), could be required to establish baseline CO conversion levels obtained with purely H₂- and CO-containing synthesis gas alone. At the lower CO conversion levels observed with FEED A compared to the BASELINE FEED, some increase in selectivity to LPG hydrocarbons was observed, although significantly greater amounts of methane and ethane were also produced. With respect to the addition of H₂ alone to the BASELINE FEED, according to the results obtained with FEED B (Example 5), use of the 3:1 H₂:CO molar ratio synthesis gas, as an LPG synthesis feed, did not cause a reduction in reaction rate, as CO conversion was comparable to that obtained with the BASELINE FEED. Nor did the addition of H₂ alone reduce the formation of CO₂, via the water-gas shift reaction. To the extent that increased H₂ concentration might drive this reaction toward CO and H₂O production, the additional H₂ also effectively displaced some CO, with the overall effect being that this additional H₂ acted essentially as an inert gas.

Surprisingly, however, compared to the BASELINE FEED, adding CO₂ and H₂ in combination to obtain FEED C (Example 6), despite causing a CO conversion deficit, resulted in nearly 70% selectivity to LPG hydrocarbons, with little or no generation of CO₂ through the LPG synthesis reactor. Whereas the selectivity to all C₁-C₄ hydrocarbons increased, the ratio of CH₄ and ethane to LPG hydrocarbons was essentially unchanged, i.e., there was no observed, disproportionate increase in these less desired C₁ and C₂ hydrocarbons. These results are therefore indicative of an unexpected increase in the yield of LPG hydrocarbons, arising from the addition of H₂ and CO₂ to synthesis gas containing predominantly H₂ and CO (e.g., having an H₂:CO molar ratio representative of synthesis gas produced by dry reforming and/or steam reforming, such as in a range from about 1.0 to about 3.0, from about 1.0 to about 2.0, or from about 2.0 to about 3.0). Such synthesis gas may be representative of a product obtained from the first stage (e.g., a reforming stage, such as a reforming/RWGS stage, or an RWGS stage), for example as a synthesis gas intermediate or portion thereof that may be withdrawn directly from a reactor used in the first stage. Importantly, a convenient source of H₂ and CO₂ useful for this addition is available, according to particular embodiments, as an H₂/CO₂-enriched fraction that may be separated from the LPG synthesis effluent and advantageously recycled to achieve the important benefits described herein. In particular, this H₂/CO₂-enriched fraction may be recycled back to the second, LPG synthesis stage and/or optionally to any locations upstream of this stage, for example as shown in the embodiment of process 1 illustrated in the FIGURE and described above. In exemplary embodiments, the H₂/CO₂-enriched fraction may be recycled by combining it with (i) the fresh makeup feed being input to the first stage, (ii) the synthesis gas intermediate or a portion thereof being input to the second stage, or some combination of (i) and (ii).

To the extent that the addition of H₂ and CO₂ was observed to cause a reduction in CO conversion, measures to compensate for this offset were investigated. From the standpoint of reaction kinetics, these measures included (a) decreasing throughput through the second, LPG synthesis stage (and/or increasing the reactor size/catalyst weight) to increase reactant residence time and/or (b) increasing the pressure in this stage to increase reactant concentrations. In terms of a second baseline case for evaluating these measures, the important consideration was the increase in selectivity to LPG hydrocarbons, obtained from FEED C (Example 6), resulting from combined H₂ and CO₂ addition, e.g., which could be realized by operating the process with a recycle stream, according to embodiments as described herein. If maintained at a higher conversion level, this increased selectivity could potentially translate to higher LPG hydrocarbon yields that are very favorable in terms of process economics. To better evaluate these possibilities, two further experiments were performed using the LPG synthesis feed corresponding to FEED C (Example 6), but with (a) a reduced normal flow rate of this feed of 97 ml/min and an increased catalyst weight of 6 grams (Example 7) and (b) additionally an increased LPG synthesis reaction pressure of 3.8 MPa (550 psi) gauge pressure (Example 8). The catalyst bed temperature was maintained at 350° C. (662° F.), and the catalyst composition was unchanged, in terms of having 25 wt-% methanol synthesis catalyst (Cu/ZnO/Al₂O₃), 75 wt-% zeolite beta. Results are summarized in Table 3 below, including CO conversion and percent carbon selectivity for various components of the LPG synthesis effluent.

TABLE 3
Variations in residence time/reaction pressure, using FEED C

| | Feed flow rate, ml/min/ | | |
|---|---|---|---|
| | Mass of MeOH synthesis catalyst and zeolite beta, g | | |
| | 165/4 | 97/6 | 97/6 |
| Example | 6 | 7 | 8 |
| WHSV, hr⁻¹ | 1.5 | 0.59 | 0.59 |
| Pressure, MPa | 2.1 | 2.1 | 3.8 |
| CO Conversion | 51.6% | 66.1% | 78.3% |
| CH₄ | 13% | 9% | 9% |
| CO₂ | 6% | 21% | 15% |
| ethane | 12% | 9% | 8% |
| propane | 40.0% | 33.7% | 34.1% |
| i-butane | 21.1% | 17.8% | 21.6% |
| n-butane | 6.9% | 8.1% | 9.8% |
| i-pentane | 1% | 1% | 2% |
| n-pentane | 0.0% | 0.0% | 0.0% |
| 2-methyl-pentane | 0.2% | 0.1% | 0.2% |
| 3-methyl-pentane | 0.1% | 0.1% | 0.1% |
| methanol | 0.0% | 0.0% | 0.0% |
| LPG hydrocarbons | 68% | 60% | 66% |
| C3 fraction of LPG | 0.59 | 0.57 | 0.52 |
| LPG Yield | 35% | 39% | 51% |

From a comparison of Examples 6 and 7, CO conversion can be increased by increasing reactant residence time (reducing throughput or weight hourly space velocity), but not necessarily with a commensurate increase in LPG yield. Rather, depending on other LPG synthesis conditions, including the specific feed composition, increased CO conversion may manifest predominantly in an increase in CO₂ production. Importantly, however, as seen from the results of Example 8, the increase in LPG synthesis reaction pressure allowed the process to operate with a single pass LPG yield exceeding 50%, due to increased conversion with reduced carbon selectivity to CO₂ and increased carbon selectivity to LPG hydrocarbons.

Overall, aspects of the invention relate to processes that utilize reforming and/or RWGS reactions to convert low value gaseous feed mixtures to LPG products, for example those comprising propane and/or butane having carbon that is derived from renewable sources, such as CH₄ and CO₂ that are the main components of biogas. Additional processing in a second reaction stage involves LPG synthesis. Those skilled in the art having knowledge of the present disclosure, will recognize that various changes can be made to these processes in attaining these and other advantages, without departing from the scope of the present disclosure. As such, it should be understood that the features of the disclosure are susceptible to modifications and/or substitutions without departing from the scope of this disclosure. The specific embodiments illustrated and described herein are for illustrative purposes only, and not limiting of the invention as set forth in the appended claims.

1. A process for producing an LPG product comprising propane and/or butane, the process comprising:
(a) in a reforming stage or an RWGS stage, contacting a gaseous feed mixture comprising predominantly (i) CH₄ and CO₂ or (ii) H₂ and CO₂ with a reforming/RWGS catalyst to produce a synthesis gas intermediate comprising an H₂/CO mixture; and
(b) in an LPG synthesis stage, converting the synthesis gas intermediate to said LPG product.

2. The process of claim 1, wherein the gaseous feed mixture comprises (i) CH₄ and CO₂ in a combined amount of at least about 75 mol-% or (ii) H₂ and CO₂ in a combined amount of at least about 75 mol-%.

3. The process of claim 1 or claim 2, wherein the gaseous feed mixture comprises one or more of CO, H₂O, and O₂, independently in an amount, or in a combined amount, of less than about 10 mol-%.

4. The process of any one of claims 1 to 3, wherein the gaseous feed mixture comprises biogas.

5. The process of any one of claims 1 to 4, wherein the LPG product is separated from an LPG synthesis effluent obtained from an LPG synthesis reactor of the LPG synthesis stage.

6. The process of claim 5, wherein the LPG product comprises propane and butane in a combined amount of at least about 80 mol-%.

7. The process of claim 5 or claim 6, wherein the gaseous feed mixture comprises a recycle portion of an H₂/CO₂-enriched fraction separated from the LPG synthesis effluent.

8. The process of any one of claims 1 to 7, wherein said converting in step (b) proceeds through a methanol synthesis reaction mechanism.

9. The process of any one of claims 1 to 8, wherein said converting the synthesis gas intermediate to said LPG product comprises contacting the synthesis gas intermediate with an LPG synthesis catalyst system comprising (i) a catalyst mixture comprising a methanol synthesis catalyst and a dehydration catalyst, or (ii) a bi-functional catalyst having a methanol synthesis-functional constituent and a dehydration-functional constituent.

10. The process of claim 9, wherein the methanol synthesis catalyst or the methanol synthesis-functional constituent comprises one or more methanol synthesis-active metals selected from the group consisting of Cu, Zn, Al, Pt, Pd, and Cr.

11. The process of claim 9 or claim 10, wherein the dehydration catalyst or the dehydration functional constituent comprises a zeolite or a non-zeolitic molecular sieve.

12. The process of any one of claims 1 to 11, wherein at least about 70% of a feed carbon content of (i) CH₄ and CO₂ or (ii) H₂ and CO₂ in the gaseous feed mixture forms the propane and/or butane.

13. An LPG product comprising propane and/or butane having a renewable carbon content of at least about 70%.

14. The LPG product of claim 13, wherein at least about 20% of a total carbon content of the LPG product is derived from CO₂.

15. The LPG product of claim 14, wherein said CO₂ is contained in biogas.

16. A process for producing an LPG product comprising propane and/or butane, the process comprising:
(a) in a reforming stage or an RWGS stage, contacting a gaseous feed mixture comprising CH₄, CO₂, and H₂ in a combined amount of at least 30 mol-% with a reforming/RWGS catalyst to produce a synthesis gas intermediate comprising an H₂/CO mixture; and
(b) in an LPG synthesis stage, contacting the synthesis gas intermediate with an LPG catalyst system to produce an LPG synthesis effluent,
(c) separating the LPG product from the LPG synthesis effluent.

17. The process of claim 16, further comprising separating one or both of (i) an H₂/CO₂-enriched fraction and (ii) a water-enriched fraction, from the LPG synthesis effluent.

18. The process of claim 17, further comprising:
recycling one or both of (i) the H₂/CO₂-enriched fraction and (ii) the water-enriched fraction to the reforming stage or the RWGS stage, or
recycling one or both of (i) the H₂/CO₂-enriched fraction and (ii) the water-enriched fraction to the LPG synthesis stage.

19. The process of any one of claims 16 to 18, wherein the gaseous feed mixture comprises biogas that is present in the gaseous feed mixture as a fresh makeup feed portion of the gaseous feed mixture.

20. The process of any one of claims 1 to 12 or 16 to 19, wherein the reforming/RWGS catalyst is disposed in a catalyst bed volume within an electrically heated reforming reactor.

21. A process for producing an LPG product comprising propane and/or butane, the process comprising contacting an LPG synthesis feed comprising H₂ and CO, and optionally CO₂, with an LPG synthesis catalyst system, said LPG synthesis catalyst system comprising a mixture of:
(i) a methanol synthesis catalyst, and
(ii) a dehydration catalyst,
to convert at least a portion of said H₂ and CO, and optionally at least a portion of said CO₂, in the synthesis gas to hydrocarbons, including propane and/or butane that are provided in the LPG product.

22. The process of claim 21, wherein the methanol synthesis catalyst and/or the dehydration catalyst comprises yttrium in elemental form or in compound form.
